(19) United States
(12) Patent Application Publication
(10) Pub. No.: US 2026/0157708 A1
(43) Pub. Date: Jun. 11, 2026

(54) SMART SCALE WITH COLOR FEEDBACK

(71) Applicant: Reestar International Limited, HONG KONG (HK)

(72) Inventor: Toby Yu, Hong Kong (HK)

(21) Appl. No.: 18/972,312

(22) Filed: Dec. 6, 2024

(51) Int. Cl. A61B 5/00; A61B 5/0205; A61B 5/26; G01G 19/44; G16H 50/30

(52) U.S. Cl. CPC A61B 5/7445; A61B 5/0205; A61B 5/26; A61B 5/702; G01G 19/44; G16H 50/30; A61B 2560/0468

(57) ABSTRACT

System apparatus, article of manufacture, method and/or computer program embodiments are provided for providing a color scheme or color-coded feedback corresponding to one or more health metrics determined by the smart scale for a user. An example method may include obtaining sensor data from one or more sensors of a smart scale; determining at least a first health metric based on the sensor data; determining a color associated with the first health metric based on color map data associated with the first health metric; and emitting a light, via a display system, in accordance with the color associated with the first health metric, the light being emitted towards one or more portions of a platform of the smart scale and causing the platform to emit light in the color associated with the first health metric.

### FIELD

This disclosure is generally directed to electronic scales and, more specifically, smart scales including platforms that output colors corresponding to a health status of a user.

### SUMMARY

Provided herein are system, apparatus, article of manufacture, method and/or computer program product embodiments (and/or combinations and/or sub-combinations thereof) for providing a color scheme or color-coded feedback corresponding to one or more health metrics determined by the smart scale for a user. In some aspects, a computer-implemented method may include obtaining sensor data from one or more sensors of a smart scale; determining at least a first health metric based on the sensor data; determining a color associated with the first health metric based on color map data associated with the first health metric; and emitting a light, via a display system, in accordance with the color associated with the first health metric, the light being emitted towards one or more portions of a platform of the smart scale and causing the platform to emit light in the color associated with the first health metric.

In some examples, a smart scale is provided for providing a color scheme or color-coded feedback corresponding to one or more health metrics determined by the smart scale for a user. The smart scale may include a platform, a display system, memory used to store data, such as computing instructions, and one or more processors coupled to the memory and configured to perform operations including obtaining sensor data from one or more sensors of a smart scale; determining at least a first health metric based on the sensor data; determining a color associated with the first health metric based on color map data associated with the first health metric; and emitting a light, via a display system, in accordance with the color associated with the first health metric, the light being emitted towards one or more portions of a platform of the smart scale and causing the platform to emit light in the color associated with the first health metric.

In some cases, a non-transitory computer-readable medium is provided for providing a color scheme or color-coded feedback corresponding to one or more health metrics determined by the smart scale for a user. In some instances, the non-transitory computer-readable medium can have instructions stored thereon that, when executed by one or more processors, may cause the one or more processors to perform operations including obtaining sensor data from one or more sensors of a smart scale; determining at least a first health metric based on the sensor data; determining a color associated with the first health metric based on color map data associated with the first health metric; and emitting a light, via a display system, in accordance with the color associated with the first health metric, the light being emitted towards one or more portions of a platform of the smart scale and causing the platform to emit light in the color associated with the first health metric.

### BRIEF DESCRIPTION OF THE FIGURES

The accompanying drawings are incorporated herein and form a part of the specification.

FIG. 1 is a block diagram illustrating an example smart scale, according to some examples of the present disclosure;

FIG. 2 is a diagram illustrating an exploded view of an example smart scale, according to some examples of the present disclosure;

FIG. 3 is a block diagram illustrating an example health and wellness environment, according to some examples of the present disclosure;

FIG. 4 is a flowchart illustrating an example method for enabling an example smart scale to output colors corresponding to health status(es) of a user, according to some examples of the present disclosure.

FIG. 5 is a diagram illustrating an example of a neural network architecture, according to some examples of the present disclosure; and

FIG. 6 illustrates an example computer system that can be used for implementing various aspects of the present disclosure.

In the drawings, like reference numbers generally indicate identical or similar elements. Additionally, generally, the left-most digit(s) of a reference number identifies the drawing in which the reference number first appears.

### DETAILED DESCRIPTION

An electronic smart scale (“smart scale” hereinafter) are weighing devices configured to measure a weight of a user and/or other various health-related metrics (e.g., weight, body fat (e.g., percentage), body mass index (BMI), skeletal muscle (e.g., percentage), fat-free mass (e.g., lbs., oz, g, etc.), subcutaneous fat (e.g., percentage), visceral fat, body water (e.g., percentage), muscle mass e.g., lbs., oz, g, etc.), bone mass (e.g., lbs., oz, g, etc.), protein (e.g., percentage), basal metabolic rate (BMR), metabolic age (e.g., years)). Despite their capabilities, existing smart scales typically present such measurements in the form of numerical values displayed on a display device associated with the smart scale. However, such numerical outputs alone may not provide sufficient guidance or context for users, who may struggle to interpret the significance of these values in relation to their health and wellness goals. Such users may find it challenging to understand the implications of the measurements or use them effectively in their health and wellness journey.

Provided herein, are a system, apparatus, device, method, and/or computer program product embodiment, and/or combinations and sub-combinations thereof (“systems and techniques” hereinafter), for a display system for a smart scale. The display system may provide a color scheme or color-coded feedback corresponding to one or more health metrics determined or generated by the smart scale for associated users. The outputted color scheme or color-coded feedback may provide the health metric(s) in a more intuitive and easily understandable format. For example, the display system may output a red indicator. The red indicator may signify that the user's BMI or body fat percentage falls within a range of BMI values associated with obesity. In another example, the display system may output a purple indicator. The purple indicator may indicate the user's body fat percentage falls within a range of body fat percentages associated with a low body fat percentage. By integrating such color-coded feedback, the smart scale enables users to gain immediate, easily interpretable insights into their health metric(s), thus simplifying the process of tracking their wellness progress and making informed health-related decisions. In some aspects, the display system may provide the color scheme or color-coded feedback corresponding to the health metric(s) along with the corresponding numerical outputs.

In some examples, the display system may be included with the smart scale. In some cases, the display system may include a client device associated with a user of the smart scale. Examples of client devices that may be associated with a user of the smart scale, include, but are not limited to, mobile phones (e.g., smartphones), set-top boxes, computers (e.g., desktop computers, laptop computers, tablet computers, etc.), televisions (TVs), Internet Protocol television (IPTV) devices or receivers, media players, displays or monitors, projectors, video game consoles, smart wearable devices (e.g., smartwatches, smart glasses, head-mounted displays (HMDs), extended reality devices (e.g., virtual reality glasses, augmented reality glasses, mixed reality glasses, virtual reality devices with video passthrough, etc.), single-board computers (SBCs) or system-on-chip (SoC) devices, and Internet-of-Things (IoT) devices, among other devices), and a remote computing system or database (e.g., cloud storage).

In some cases, users may inconsistently use the smart scale (e.g., users may forget to or fail to regularly weigh themselves). This inconsistency can hinder the effectiveness of health and wellness tracking, as infrequent measurements limit the ability to accurately monitor trends and changes in the user's health metric(s). Smart scales generally do not provide reminders or prompts to the users to weigh or measure themselves on the smart scales.

Provided herein are systems and techniques for a reminder system for a smart scale. The reminder system may prompt or remind the user to weigh in or measure themselves using the smart scale. In some examples, the reminder system may communicate with the display system of the smart scale to output the prompt or reminder. In some instances, the prompt or reminder may be a visual output, such as a color, text, images, videos, etc., and/or an audio output. In some cases, the reminder system may communicate with the display system to output the prompt or reminder.

In some aspects, the reminder system may provide the prompt or reminder while the smart scale is in a low-power mode (e.g., smart scale may operate with reduced functionality to conserve battery life). In some examples, the reminder system may provide the prompt or reminder at a predetermined time. The predetermined time may bet set by a user (e.g., via a corresponding user interface(UI)) and/or be determined by the reminder system based on behavioral data (e.g., the scale may analyze historical weigh-in times to determine a probable time when the user is likely to be near the scale). In some examples, the reminder system may provide the prompt or reminder by detecting when the user is near the smart scale and/or within a proximity distance threshold of the smart scale. The reminder system may use one or more sensors, such as a microphone and/or proximity sensors (e.g., infrared (IR) sensors, ultrasonic sensors, passive infrared (PIR) sensors, radar sensors, etc.), and/or location-based technologies (e.g., Global Positioning System (GPS), Wi-Fi, Bluetooth, near field communication (NFC), etc.) to detect when the user or a client device of the user is near the smart scale or within a proximity distance threshold of the smart scale. The reminder system may enhance engagement between the smart scale and the user and help maintain consistent weigh-ins and health metric(s) determinations, thereby improving the reliability and continuity of health and wellness tracking.

In some cases, the smart scales may be positioned in one location, such as a bathroom. In such cases, the smart scales may be inactive when not performing weigh-ins or determining health metric(s). During nighttime hours or when there are low light conditions in those locations, users may navigate to such locations, which can be disorienting and unsafe under those low light conditions. Activating standard lights in such locations may provide excessive illumination, often causing discomfort and temporary visual impairment due to sudden exposure to bright light. In some instances, the abrupt change in lighting can disrupt the user's natural sleep cycle and increase the risk of stumbling or tripping.

Provided herein are systems and techniques for a low-light system for a smart scale. In some examples, the low-light system may determine whether the smart scale is in an environment with low-light conditions. The low-light system may determine when a user is near the smart scale (e.g., within a proximity distance threshold of the smart scale). Based on the low-light system determining the user is near the smart scale and the smart scale is in an environment with low-light conditions, the low-light system may provide low-level, eye-friendly illumination (e.g., 0.1 lux to 100 lux). The low-light system may communicate with the display system to emit the low-level, eye-friendly light.

In some aspects, the low-light system may use one or more sensors, such as a microphone and/or proximity sensors (e.g., infrared (IR) sensors, ultrasonic sensors, passive infrared (PIR) sensors, radar sensors, etc.), and/or location-based technologies (e.g., Global Positioning System (GPS), Wi-Fi, Bluetooth, near field communication (NFC), etc.) to detect or determine whether the user or a client device of the user is near the smart scale or within a proximity distance threshold of the scale. Based on the user being near the smart scale, the low-light system may cause the display system of the smart scale to output a low-level, eye friendly illumination.

In some cases, the low-light system may cause the display system to output a light in a low-level intensity range with an output power between 0.01-1.0 watts, based on the low-light system determining a user is near the smart scale (e.g., within a proximity distance threshold). The described power range ensures that the light remains gentle, minimizing retinal stimulation and reducing the risk of eye strain or potential harm to the eyes of the user.

In some instances, the low-light system may cause the display system to output a light in a low-level intensity range with a wavelength range of approximately 500 to 600 nanometers, corresponding to warm colors, such as yellow, amber, or soft orange. The warm colors may be less likely to interfere with melatonin production or disrupt the user's sleep cycle, compared to cooler, blue-spectrum light (below 480 nanometers). In some examples, the warm colored, low-light intensity light output may enable the eyes of a user near the smart scale in a low-light condition to quickly adapt to the low-light conditions without causing shock or discomfort. In some examples, the warm light provides sufficient illumination for the user to safely navigate the environment with the low light level conditions the smart scale is in, reducing the risk of accidents while ensuring a non-intrusive experience that does not disturb the user's night vision or sleep.

In some cases, multiple users may use a smart scale. In such cases, smart scales may not be able to track health metrics for each of the multiple users. As such, each of the multiple users may struggle to monitor trends and changes in their health and wellness journey. Provided herein are systems and techniques for a profile management system for a smart scale. The profile management system may enable users to set up and configure a corresponding user profile for the smart scale. Additionally, or alternatively, the profile management system may enable the smart scale to store determined and measured health metrics of each of the multiple users and associate the health metrics to a corresponding user profile.

In some examples, the profile management system may enable the users to manually select a user profile the smart scale may associate the measured and determined health metrics to. In some cases, the profile management system may automatically select a user profile for a user using or about to use the smart scale. In some instances, the profile management system may automatically select a user profile for a user using or about to use the smart scale by identifying the user using the smart scale or who is about to use the smart scale. The profile management system may cause the smart scale to associate the corresponding determined or measured health metrics to the selected user profile. That way, the profile management system may enable the smart scale to automatically switch to the appropriate user profile without requiring manual input from a user.

In some aspects, the profile management system may use location-based technologies (e.g., Global Positioning System (GPS), Wi-Fi, Bluetooth, near field communication (NFC), etc.) to identify a user using or about to use the smart scale. For example, based on location data of client devices of users, the profile management system may determine client devices of users that are the nearest to the smart scale. Based on the device data of the client device of the users (e.g., including identifying information associated with the computing device of the user and/or the user), the profile management system may determine the identity of the user associated with the client device that is nearest to the smart scale. The profile management system may automatically select a corresponding profile of the identified user.

In some instances, the profile management system may identify a user using the smart scale based on characteristics of the user standing on the smart scale. In such instances, the profile management system may select a corresponding user profile to store determined or measure heath metrics of the user to. In some cases, the characteristics of the user may include the health metrics of the user that the smart scale determines or measures. Additionally, or alternatively, the characteristics of the user may include gait and stance information of the user. The profile management system may use sensor data generated from the sensor(s) of the smart scale to determine the gait and stance information of the user, including but not limited to a pattern of balance, stance, width, and pressure points when stepping onto the scale. The profile management system may automatically select a corresponding user profile of the identified user. In some aspects, the profile management system may use one or more artificial intelligence (AI) or machine learning (ML) algorithms or models (e.g., convolutional neural networks (CNNs), Long Short-Term Memory (LSTM) networks, Support vector Machines (SVM), and/or Random Forests (RF)) to determine the gait information of the user standing on a smart scale and/or an identify of a user standing on a smart scale based on the gait and stance information of the user and/or the measured and determined heath metrics of the user.

The present disclosure recognizes that the use of personal information data can be used to the benefit of users. For example, personal information data can be used to better understand user behavior, facilitate and measure the effectiveness of applications and delivered digital content. Accordingly, use of such personal information data enables calculated control of the delivered digital content. For example, the system can reduce the number of times a user receives certain content and can thereby select and deliver content that is more meaningful to users. Such changes in system behavior improve the user experience. Further, other uses for personal information data that benefit the user are also contemplated by the present disclosure. The present disclosure further contemplates that the entities responsible for the collection, analysis, disclosure, transfer, storage, or other use of such personal information data should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after the informed consent of the users. Additionally, such entities would take any needed steps for safeguarding and securing access to such personal information data and ensuring that others with access to the personal information data adhere to their privacy and security policies and procedures. Further, such entities can subject themselves to evaluation by third parties to certify their adherence to widely accepted privacy policies and practices.

Despite the foregoing, the present disclosure also contemplates embodiments in which users selectively block the use of, or access to, personal information data. Moreover, the present disclosure includes mechanisms which can be implemented to protect the privacy of users and anonymize data collected. Although the present disclosure may cover use of personal information data to implement one or more various disclosed embodiments, the present disclosure also contemplates that the various embodiments can also be implemented without the need for accessing and/or reporting such personal information data and/or with protections to maintain the user's privacy. The various embodiments of the present technology are not rendered inoperable due to the lack of all or a portion of such personal information data.

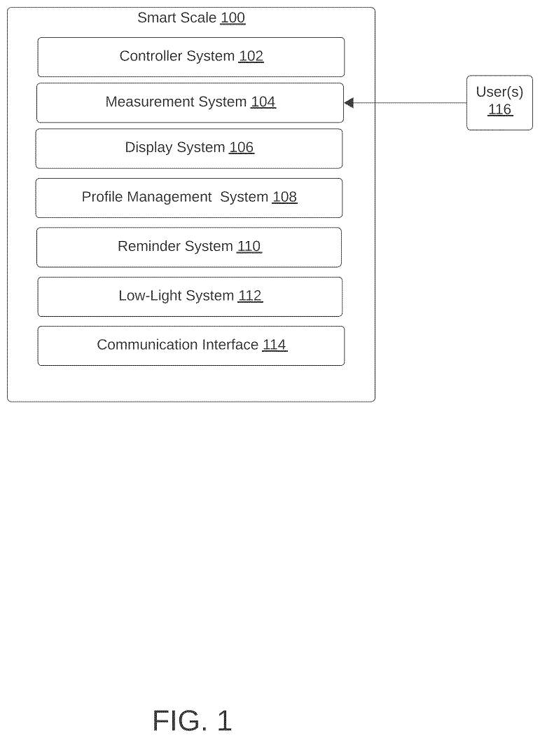

Various embodiments and aspects of this disclosure may be implemented using and/or may be part of smart scale 100 shown in FIG. 1. It is noted, however, that smart scale 100 is provided solely for illustrative purposes and is not limiting. Examples and embodiments of this disclosure may be implemented using, and/or may be part of, environments different from and/or in addition to the smart scale 100, as will be appreciated by persons skilled in the relevant art(s) based on the teachings contained herein. An example of the smart scale 100 shall now be described.

### Smart Scale

Referring to FIG. 1, example smart scale 100 may be configured to perform any of the example processes described herein. In some examples, smart scale 100 may provide a color scheme or color-coded feedback corresponding to health metric(s) determined or generated by smart scale 100. In some cases, smart scale 100 may, amongst other things determine health metric(s) for users, provide the determined health metric(s) to the users, provide, to the users a color scheme or color-coded feedback corresponding to the health metric(s) associated with the users, enable users to customize color maps, provide prompts or reminders for users to weigh in or measure themselves using smart scale 100, determine behavior data or information, enable users to set a time for the reminder(s/prompt(s), and/or provide a low-level, eye friendly illumination (e.g., 0.1 lux to 100 lux) for locations with low light conditions the users and smart scale 100 are in.

In some examples, smart scale 100 may include, be part of, and/or be implemented by one or more hardware and/or software systems, such as, for example and without limitation, one or more server computers, datacenters and/or datacenter devices, cloud computing infrastructure devices/components, software containers, virtual machines, computer devices, cloud application services, microcontroller units and/or any other computing systems. As illustrated in FIG. 1, smart scale 100 may include controller system 102, measurement system 104, display system 106, profile management system 108, reminder system 110, low-light system 112, and communication interface 114. In some aspects, controller system 102 may each include or represent one or more artificial intelligence (AI) or machine learning (ML) processes, algorithms or models, such as but not limited to, convolutional neural networks (CNNs), Long Short-Term Memory (LSTM) networks, Support vector Machines (SVM), Random Forests (RF), and/or any other AI/ML models that may determine one or more health metrics, predict health status of users based on health metrics over time or a period of time, behavioral information (such as device usage), etc.

In some cases, one or more processors of smart scale 100 may executed controller system 102 to implement or perform any of the example processes described herein, such as, but not limited to, determining health metric(s) for users, providing the determined health metric(s) to the users, providing, to the users a color scheme or color-coded feedback corresponding to the health metric(s) associated with the users, enabling users to customize color maps, providing prompts or reminders for users to weigh in or measure themselves using smart scale 100, determining behavior data or information, enabling users to set a time for the reminder(s/prompt(s), and/or providing a low-level, eye friendly illumination (e.g., 0.1 lux to 100 lux) for locations with low light conditions the users and smart scale 100 are in.

In some aspects, controller system 102 may provide a color scheme or color-coded feedback corresponding to health-related measurements or metric to users of smart scale 100 by determining one or more health-related measurements or metrics (“health metrics” hereinafter). In such aspects, controller system 102 may communicate with measurement system 104 to obtain signals or sensor data generated by measurement system 104. Controller system 102 may determine the health metric(s) of a user using smart scale 100 based on the signals or sensor data generated by the measurement system 104. Examples of health metric(s), include, but are not limited to, weight, body fat (e.g., percentage), body mass index (BMI), skeletal muscle (e.g., percentage), fat-free mass (e.g., lbs., oz, g, etc.), subcutaneous fat (e.g., percentage), visceral fat, body water (e.g., percentage), muscle mass e.g., lbs., oz, g, etc.), bone mass (e.g., lbs., oz, g, etc.), protein (e.g., percentage), basal metabolic rate (BMR), metabolic age (e.g., years). Controller system 102 may provide a color scheme or color-coded feedback corresponding to a user based on the determined health metric(s).

In some instances, measurement system 104 may include one or more components configured to generate the signals and/or sensor data controller system 102 uses to determine the health metric(s) of a user. The component(s) of measurement system 104 may include, but are not limited to, a platform, one or more sensors, one or more electrodes, and one or more electrical current generators. In some examples, the platform may enable users to stand on the platform to have their heath metric(s) determined. In some cases, the platform may be formed out of material with high mechanical strength to at least withstand a weight of a user standing on the platform (e.g., approximately at most of 500 pounds or 226 kilograms). In some aspects, the sensor(s) may be operatively coupled to the platform. The sensor data generated by the sensor(s) may indicate a weight of a user standing on the platform. Controller system 102 may process the sensor data to determine a health metric, such as a weight, of the user standing on the platform. In some instances, the electrode(s) may be operatively coupled to the platform to obtain signals from a user standing on the platform that controller system 102 may use to determine health metric(s) of the user. In some examples, the electrode(s) may be included or embedded in the platform. In such examples, the platform may be formed out of tempered and conductive glass (e.g., indium tin oxide) to enable electrical signals, such as current, to pass through to a user standing on the platform. In some cases, the one or more electrical current generators may be electrically coupled to the electrodes to provide the electrical signal to the electrodes. The electrical signal that passes through the body of the user and from the electrodes may then be received by the electrodes. In some aspects, controller system 102 may process the sensor data and/or the electrical signals received from the electrodes to determine additional health metric(s) of the user standing on the platform (e.g., body fat (e.g., percentage), body mass index (BMI), skeletal muscle (e.g., percentage), fat-free mass (e.g., lbs., oz, g, etc.), subcutaneous fat (e.g., percentage), visceral fat, body water (e.g., percentage), muscle mass e.g., lbs., oz, g, etc.), bone mass (e.g., lbs., oz, g, etc.), protein (e.g., percentage), basal metabolic rate (BMR), metabolic age (e.g., years). Controller system 102 may provide a color scheme or color-coded feedback corresponding to a user based on the determined health metric(s)). In some instances, controller system 102 may perform bioelectrical impedance analysis (BIA) to determine the additional health metric(s) of the user standing on the platform.

In some aspects, controller system 102 may process a color map data and the heath metric(s) of a user to provide, to the user, a color scheme or color-coded feedback corresponding to the health metric(s). As described herein, the color map data may identify one or more color maps. Each color map may be associated with a type of health metric (e.g., weight, body fat (e.g., percentage), body mass index (BMI), skeletal muscle (e.g., percentage), fat-free mass (e.g., lbs., oz, g, etc.), subcutaneous fat (e.g., percentage), visceral fat, body water (e.g., percentage), muscle mass e.g., lbs., oz, g, etc.), bone mass (e.g., lbs., oz, g, etc.), protein (e.g., percentage), basal metabolic rate (BMR), metabolic age (e.g., years)). In some instance, the color map data may identify, for each color identified in each color map, a corresponding health metric value and/or a range of health metric values. Each heath metric value and/or range of health metric values identified in a color map may correspond to a particular health status. Examples of the health status, may include, but are not limited to, low or underweight, slightly low or underweight, average weight or standard weight, slightly high weight, high weight, low BMI, slightly low BMI, standard BMI, slightly high BMI, high BMI, low body fat percentage, slightly low body fat percentage, standard fat percentage, slightly high fat percentage, high fat percentage, low subcutaneous fat percentage, slightly low subcutaneous fat percentage, standard subcutaneous fat percentage, slightly high subcutaneous fat percentage, high subcutaneous fat percentage, low visceral fat, slightly low visceral fat, standard low visceral fat, slightly high low visceral fat, high low visceral fat, low body water percentage, slightly low body water percentage, standard body water percentage, slightly high body water percentage, high body water percentage, hydrated, dehydrated, low muscle mass percentage, slightly low muscle mass percentage, standard muscle mass percentage, slightly high muscle mass percentage, high muscle mass percentage, low bone mass, slightly low standard bone mass, standard bone mass, slightly high bone mass, high bone mass, low protein mass percentage, slightly low protein mass percentage, standard protein mass percentage, slightly high protein mass percentage, high protein mass percentage, low basal metabolic rate (BMR), slightly low BMR, standard BMR, slightly high BMR, high BMR, excellent metabolic age, and/or high metabolic age. For example, color map data may identify a color map for BMI. The color map may identify a variety of colors and each color may correspond to a particular range of BMI values. Each range of BMI values may correlate to a particular health status related to BMI, such as low BMI, slightly low BMI, standard BMI, slightly high BMI, and high BMI.

In some examples, controller system 102 may determine a color of an associated color map that correlates to a health metric determined by controller system 102 based on the color map data and the health metric. For example, controller system 102 determines a BMI value for a user based on electrical signals and/or sensor data generated by measurement system 104. Based on the color map data and the BMI value, controller system 102 may identify a color map associated with the health metric type, BMI. Based on the color map and the BMI value, controller system 102 may identify a color associated with a range of health metric values that the BMI value of the user is within or a color associated with a health matric value that matches the BMI value of the user. Controller system 102 may provide the identified color to the user (e.g., purple).

In some cases, controller system 102 may provide a color scheme or color-coded feedback corresponding to health metric(s) to users of smart scale 100 via display system 106 of smart scale 100. For example, controller system 102 may communicate with display system 106 to output a color corresponding to a health metric of a user controller system 102 determined. In some aspects, display system 106 may include a light source. In some examples, the light source may include one or more LEDs (e.g., an array of LEDs, such as a light strip). Controller system 102 may independently adjust or configure parameters of each of LED(s) of display system 106. Examples of parameters controller system 102 may adjust, include but are not limited to, the brightness and color. For example, controller system 102 may configure the LED(s) RGB (Red, Green, Blue) to provide a wide spectrum of colors via display system 106 (e.g., cyan, magenta, yellow, purple, orange, etc.).

For example, user 116 steps onto a platform of measurement system 104. Based on the electrical signals and/or sensor data generated by the measurement system 104 (e.g., via the electrodes and/or pressure sensor data) controller system 102 may determine a health metric, such as muscle mass percentage. Based on color map data, controller system 102 may identify a color map associated with the type of health metric controller system 102 determined, such as a color map associated with muscle mass percentage. Based on the color map and the determined health metric of the user, controller system may adjust or configure the output of the light source of display system 106, such as the RGB of one or more LEDs according to the color map and the determined health metric of the user. For example, if the value of the determined muscle mass percentage is within a range of values identified in the color map corresponding to orange, controller system 102 may adjust the parameters of the LED(s), such as the RGB), so that the LED(s) output the color orange. The color orange and the corresponding range of values may correspond to a high level of muscle mass percentage.

In some examples, the color map of the color map data may be configured by a user. In such examples, a user may use a user interface (UI) associated with smart scale 100 to configure the color map. For example, the UI may include one or more interface elements that enable the user to select, for a color map, particular colors for each health metric value or range of health metric values (e.g., purple for a range of health metric values of BMI related to obesity or green for a range of health metric values of BMI related to obesity).

In some instances, display system 106 may include a backlight board component (e.g., edge-lit backlight board, direct-lit backlight board, etc.). The backlight board component may be configured to ensure even illumination of the light emitted by a light source of the display system, such as the LED(s). In some instances, the backlight board may include a reflection board, light guide plate, reflective films, light diffusing film and/or, in some instances a protective plate. As described herein, the reflection board redirects light emitted from the light source upward enhancing the efficiency of light utilization, the light guide plate is configured to distribute the light emitted from the light source uniformly across a surface, such as a surface formed out of transparent, the reflective film(s) may enhance light distribution and minimize light leakage emitted from the light source, the light diffusing film scatters the light emitted from the light source evenly, reducing hotspots and ensuring that the color output is consistent across the entire display area, and the protective plate may protect the other components of the backlight board while maintaining optical clarity.

In some examples, the light source and/or the backlight board may be coupled to a platform of measurement system 104. In such examples, the platform may be formed out of a transparent or translucent material, such as a conductive glass or acrylic material. The light source, such as the LED(s) and/or the backlight board may output light in one or more colors that illuminate one or more portions of the surface of the platform. In some instances, the backlight board may be positioned underneath the platform (e.g., the reflection board may be positioned at the base of the backlight board and reflects the light emitted from the light source towards the platform, the light guide path is positioned above the reflection board, the reflective films may be layered above the light guide plate, the light diffusing films may be positioned above the reflective films, and the protective plate is positioned above the backlight board). For instance, when a user steps onto the platform, controller system 102 may cause the light source of display system 106 to illuminate the platform red. Based on a corresponding color map, the color red may indicate the user's heath metric indicates a body fat percentage falls within a range of body fat percentages associated with a high body fat percentage. Alternatively, a soft green glow may indicate that the user's health metrics fall within an optimal range as indicated by the color map data. By coupling the display system to the platform, the smart scale provides an intuitive, ambient visual feedback mechanism that enhances the user experience and offers real-time, actionable health insights.

In some cases, the backlight board may enable controller system 102 to provide two or more color schemes or color-coded feedback at once on a platform of smart scale 100. Each of the two or more color schemes may correspond to a health metric of a user controller system 102 may have determined. In such cases, the platform may include two or more zones or areas. Controller system 102 may cause display system 106 to output different colors for each of the two or more zones. In some examples, the backlight board may include one or more components that enable controller system 102 to provide two or more color schemes or color-coded feedback at once on the platform. Examples of the component(s) include, but are not limited to a light guide plate configured with microstructures that direct light emitted from the light source even across the surface to prevent color mixing or uneven illumination, two or more segmented or zoned diffusing films that may enable and maintain color differentiation between adjacent areas allowing the platform to display multiple colors simultaneously without the colors mixing or blending, and/or two or more optical barriers or dividers that separate the zones, prevent potential color overlap between each zone, and maintain sharp color boundaries between the zones.

For example, user 116 may step onto the platform of measurement system 104. The backlight bard of display system 106 may divide or segment the platform into two zones—a first zone associated with one health metric and a second zone associated with another health metric. Controller system 102 may determine a BMI value and a skeletal muscle percentage based on electrical signals and/or sensor data generated by measurement system 104. Based on color map data, controller system 102 may identify a color map associated with the BMI value for the user and a color map associated with the skeletal muscle percentage of the user. Based on the color map associated with the BMI value, controller system 102 may determine a corresponding color, such as green (e.g., indicating a standard BMI). Based on the color map associated with the skeletal muscle percentage, controller system 102 may determine a corresponding color, such as yellow (e.g., indicating a low skeletal muscle mass). Controller system 102 may cause display system 106, such as LED(s), to illuminate each of the first zone and the second zone in accordance with the identified colors. For instance, controller system 102 may cause display system 106 to illuminate the first zone in accordance with the color identified for the health metric associated with the BMI of the user (e.g., green), while controller system 102 may cause display system 106 to illuminate the second zone in accordance with the color identified for the health metric associated with the skeletal muscle percentage of the user (e.g., yellow), or vice versa.

In some aspects, display system 106 may include a display device, such as an LCD, LED or OLED screen. The display device may output numerical measurements associated with the health metrics determined by controller system 102. For example, user 116 may step onto the platform of measurement system 104. Controller system 102 may determine one or more health metrics, such as health metrics associated with weight and BMI, based on electrical signals and/or sensor data generated by measurement system 104. The display device may output numerical measurements associated with health metric(s) determined by controller system 102, such as a numerical value associated with the determined user's weight and a numerical value associated with the determined user's BMI.

In some examples, controller system 102 may provide a color scheme or color-coded feedback corresponding to health metrics that reflect a user's progress over time or a period of time. The health metrics that reflect the user's progress over time may identify increases or decreases in specific health metrics and/or overall progress towards a health goal a user may have defined (e.g., via a UI associated with smart scale 100). The color map data may include one or more color maps that each may be associated with a type of health metric over time. In such examples, controller system 102 may store health metric(s) determined for a user in one or more data storage systems associated with smart scale 100. Examples of the data storage system(s) include, but are not limited to, on-device memory of smart scale 100, a memory or storage device of a client device of a user (e.g., mobile phones (e.g., smartphones), set-top boxes, computers (e.g., desktop computers, laptop computers, tablet computers, etc.), televisions (TVs), Internet Protocol television (IPTV) devices or receivers, media players, displays or monitors, projectors, video game consoles, smart wearable devices (e.g., smartwatches, smart glasses, head-mounted displays (HMDs), extended reality devices (e.g., virtual reality glasses, augmented reality glasses, mixed reality glasses, virtual reality devices with video passthrough, etc.), single-board computers (SBCs) or system-on-chip (SoC) devices, and Internet-of-Things (IoT) devices, among other devices), and a remote computing system or database (e.g., cloud storage). Controller system 102 may obtain from the one or more data storage systems the stored health metrics determined for a user and corresponding timestamps (“historical health information” hereinafter). Controller system 102 may determine health metrics that reflect a user's progress over time based on the historical health information of the user. Based on the health metrics that reflect a user's progress over time and a corresponding color maps, controller system 102 may identify a corresponding color to output via display system 106 as described herein.

For example, controller system 102 may determine health metric(s) for user 116 that consistently uses smart scale 100, such as weight, and body fat percentage based on electrical signals and/or sensor data generated by measurement system 104. Controller system 102 may obtain historical health information associated with the determined health metric(s). Based on color map data, controller system 102 may identify a color map associated with the determined health metric(s) and/or the corresponding historical health information, such as a color map associated with the BMI value for the user over a period of time and a color map associated with the body fat percentage for the user over a period of time. Based on the color map(s) associated with the health metric(s) for user 116, controller system 102 may determine a corresponding color, such as green (e.g., indicating a decreased BMI value) or red (e.g., indicating an increase in body fat percentage). Controller system 102 may cause display system 106, such as LED(s), to illuminate one or more portions of a platform of smart scale 100.

In some examples, controller system 102 may provide a UI associated with smart scale 100 to inform a user (e.g., user 116) of the health metric(s) controller system 102 determined and/or health metrics that reflect the user's progress over time. In some instances, controller system 102 may the display device of display system 106 to display the UI. In some cases, controller system 102 may provide the UI to a client device of the user. The client device may display the UI to inform the user of the health metric(s) controller system 102 determined and/or health metrics that reflect the user's progress over time.

In some cases, the health metrics that reflect a user's progress over time may include patterns and predictions about the user's (e.g., user 116) health and wellness journey associated with the health metric(s) determined by controller system 102. In such cases, based on historical health information of a user and/or determined corresponding health metric(s), controller system 102 may use one or more AI/ML models or algorithms to identify patterns, and/or predict or determine a trajectory of a user's health and wellness journey associated with the health metric(s) determined by controller system 102. Controller system 102 may obtain corresponding AI/ML dataset(s) including one or more parameters of the trained AI/ML model(s) or algorithm(s) that may be stored in the data storage system(s) associated with smart scale 100. As described herein, such trained AI/ML model(s) or algorithm(s) may be trained historical health information of the users to identify patterns and make predictions about the user's health and wellness journey. In some instances, controller system 102 may cause a display device of display system 106 to display information of the determined patterns and predictions about the user's health and wellness journey. In some examples, controller system 102 may cause a UI associated with smart scale 100 to display information of the determined patterns and predictions about the user's health and wellness journey. In some instances, controller system 102 may provide the UI to a client device of a user, such as user 116. The client device may display the UI including information of the determined patterns and predictions about the user's health and wellness journey.

In some instances, the data storage system(s) may store profile information for user(s), such as user(s) 116, of smart scale 100. The profile information may include identifying information of a corresponding user (e.g., a name, contact information (e.g., an address, a phone number, an email address, etc.), one or more governmental identifiers (e.g., a driver's license number, a social security number, etc.), and/or demographic information that characterizes the corresponding user, such as, for example, an age, gender, location, etc.), device data of a client device of the user that has communicated with smart scale 100 (e.g., a device identifier), and/or historical health information and corresponding timestamps. In some examples, the determined health metrics of the users, and/or the color map data may be stored in one or more data storage systems associated with smart scale 100.

In some cases, profile management system 108 may enable users, such as users 116, to create, configure, and manage individual user profiles, each containing associated profile information. In some examples, profile management system 108 may provide a UI associated with smart scale 100 to the users. The UI may include one or more interface elements that enable the users to create, configure, and manage corresponding user profiles. For example, the interface element(s) may enable the users to provide profile information as described herein when creating a profile, such as identifying information of a corresponding user (e.g., a name, contact information (e.g., an address, a phone number, an email address, etc.), one or more governmental identifiers (e.g., a driver's license number, a social security number, etc.), and/or demographic information that characterizes the corresponding user, such as, for example, an age, gender, location, etc.). In some examples, profile management system 108 may associate device data of a client device (e.g., a device identifier) of a user using smart scale 100. In some instances, the UI may include interface element(s) that enable the user to identify health goals. For example, if the user has paired or is communicating with smart scale 100 with an associated client device, profile management system 108 may obtain the device data of the client device and associate the device data to an associated user profile. In some instances, controller system 102 may cause the UI to be displayed by a display device of display system 106. Additionally, or alternatively, controller system 102 may provide the UI to a client device of a user, such as user 116. The client device may display the UI.

In some aspects, profile management system 108 may be enable controller system 102 to associate or store determined and measured health metrics of each of the users and corresponding timestamps to corresponding user profiles. In such aspects, profile management system 108 may identify a user and corresponding user profile to store or associate the associated determined and health metrics. In some instances, profile management system 108 may identify the user using or about to use smart scale 100 and corresponding user profile based on one or more provided inputs from the user. For example, user 116 may manually select their own user profile via the display device of display system 106. Alternatively, user 116 may manually select their own user profile via a client device of the user communicating or paired with smart scale (e.g., via one or more interface elements of a UI associated with smart scale 100 that is displayed by the client device). The selected user profile may indicate to profile management system 108 a user profile to associate health metrics determined by controller system 102.

In some examples, profile management system 108 may automatically identify a user, such as user 116, using or about to use smart scale 100 (e.g., standing on a platform of measurement system 104) and corresponding user profile. Profile management system 108 may each include or represent one or more artificial intelligence (AI) or machine learning (ML) processes, algorithms or models, such as but not limited to, convolutional neural networks (CNNs), Long Short-Term Memory (LSTM) networks, Support vector Machines (SVM), Random Forests (RF), and/or any other AI/ML models that may determine or identify a user using or about to use smart scale 100 (e.g., stepping on a platform of measurement system 104) based on a determined a pattern of balance, stance, width, and pressure points of the user when stepping onto the platform.

In some aspects, profile management system 108 may identify a user using or about to use smart scale 100 and corresponding user profile based on a proximity of an associated client device to smart scale 100. In such aspects, profile management system 108 may use location-based technologies, such as Global Positioning System (GPS), Wi-Fi, Bluetooth, and Near Field Communication (NFC), to detect the proximity of a client device associated with the user, such as a smartphone, smartwatch, or other wearable device, relative to smart scale 100, based on location data of the client device. Profile management system 108 may determine which of the client device(s) is nearest to smart scale 100 at a given time based on location data of the client device(s). In some instances, a client device of a user, such as the nearest client device to smart scale 100, may transmit device data associated with the client device to smart scale 100, either separate from the location data, with the location data or including in the location data. As described herein, the device data may include identifying information about the client device, such as a unique device identifier (e.g., MAC address, Bluetooth UUID). Profile management system 108 may identify a user profile that includes device data matching or similar to the device data received from the client device nearest to smart scale 100. In some instances, profile management system 108 may determine the identity of the user associated with the client device closest to smart scale 100 based on the identified user profile that includes device data matching or similar to the device data received from the client device. In some cases, profile management system 108 may employ a hierarchical detection protocol, prioritizing client devices with stronger signals (e.g., higher RSSI values in Bluetooth) or more precise location data (e.g., NFC detection) to accurately identify the user and/or client device closest to smart scale 100.

Profile management system 108 may automatically select user profile associated with client device nearest to smart scale 100, without requiring manual input. Health metric(s) of the associated user determined by controller system 102 may be stored or associated with the selected user profile. For example, if profile management system 108 detects a nearby smartphone belonging to User A, identified through its Bluetooth signal strength and device identifier, profile management system 108 may automatically load User A's user profile and attribute the upcoming health metrics controller system 102 determines to that user profile.

In some cases, profile management system 108 may identify a user using the smart scale based on characteristics of the user standing on the smart scale. Based on the identification, profile management system 108 may automatically select a corresponding user profile for storing associated heath metric(s) determined or measured by controller system 102. In some instances, the characteristics used for identification may include the health metric(s) measured or determined by controller system 102. Additionally, or alternatively, the characteristics may include gait and stance information associated with the user. In some examples, profile information of users may include gait and stance information.

For example, profile management system 108 may provide a UI associated with smart scale 100. The UI may include one or more interface elements that enable a user to create a profile. Additionally, or alternatively, the interface element(s) may enable the user to calibrate, record or store, gait information of the user to a user profile the user is creating. Such interface element(s) may prompt a user to stand on a platform of measurement system 104. One or more sensors (e.g., pressure sensors, load cells, or capacitive sensors, etc.) of measurement system 104 may generate sensor data that indicates a gait and stance of a user, including but not limited to, the balance, stance width, pressure distribution, and/or pressure points of a user standing on a platform of measurement system 104. Profile management system 108 may determine the gait and stance of the user based on the sensor data. Profile management system 108 may store information of the determined gait and stance or the gait and stance information to the user profile the user is creating.

In some cases, profile management system 108 may identify a user standing on the platform based on the profile information of the user including the gait information. For example, the sensor(s) (e.g., pressure sensors, load cells, or capacitive sensors, etc.) of measurement system 104 may generate sensor data associated with a user, such as user 116, standing on a platform of measurement system 104. Profile management system 108 may process the sensor data to determine a gait and stance of a user, including but not limited to, the balance, stance width, pressure distribution, and/or pressure points of a user standing on a platform of measurement system 104. Profile management system 108 may identify a user profile with similar gait and stance information as the determined gait and stance. In some instances, profile management system 108 may automatically select the identified user profile to store health metric(s) controller system 102 determines for the associated user.

In some cases, controller system 102 may determine health and wellness related recommendations (“recommendations” hereinafter) based on health metric(s) of a user determined by controller system 102. The recommendations may provide a form of personalized feedback for the user during their health and wellness journey. In some examples, controller system 102 may process health metric(s) controller system 102 determined for a user. The health metric(s) may include health metric(s) previously determined by controller system 102 and/or stored in one or more data storage systems associated with smart scale 100 (e.g., on-device memory of smart scale 100, on-device memory and/or storage devices of a client device of the user, and/or remote computing systems or databases). In some instances, controller system 102 may use one or more trained AI/ML models or algorithms to determine the recommendations based on the health metric(s) of the user. In such instances, controller system 102 may obtain corresponding AI/ML dataset(s) including one or more parameters of the trained AI/ML model(s) or algorithm(s) that may be stored in the data storage system(s) associated with smart scale 100. As described herein, the trained AI/ML model(s) or algorithm(s) may be trained using datasets comprising various health metrics and corresponding wellness guidelines, enabling the models to identify patterns and correlations between different health indicators.

For example, user 116 may step onto the platform of measurement system 104. Controller system 102 may determine multiple health metrics associated with the user, including a BMI value, a muscle mass percentage and a body fat percentage based on electrical signals and/or sensor data generated by measurement system 104. Based on the health metrics of the user, controller system 102 may determine a recommendation. For instance, based on the health metrics of the user indicating the user has a high BMI value, a low muscle mass percentage and a high body fat percentage, controller system 102 may determine a recommendation suggesting the user should increase aerobic exercise and/or monitor their caloric intake to at least reduce body fat levels. In another instance, based on the health metrics of the user indicating the user has a high BMI value, a high muscle mass percentage and a low body fat percentage, controller system 102 may determine a recommendation encouraging the user's current health and/or provide a warning about the potential impact of higher body weight on joint health. In such an instance, the recommendation may further include a suggestion for low-impact exercises like swimming or cycling. Controller system 102 may cause a display device of display system 106 to output the recommendation.

In some instances, controller system 102 may generate and output, via display system 106, a recommendation based on the health metrics that reflect a user's (e.g., user 116) progress over time. For example, controller system 102 may determine a health metric associated with weight for user 116 that consistently uses smart scale 100 based on electrical signals and/or sensor data generated by measurement system 104. Controller system 102 may obtain historical health information related to the weight for user 116. Based on historical health information related to the weight for user 116 and/or the determined health metric associated with the weight of user 116, controller system 102 may determine a recommendation (e.g., a recommendation indicating weight of user 116 is increasing and/or user 116 should consider adjusting exercise routine(s) and/or dietary intakes).

In some aspects, controller system 102 may provide real-time recommendations and/or information of the determined health metric(s) of a user (e.g., user 116) with or without network connectivity. In some cases, controller system 102 may implement a distributed processing architecture, leveraging the computational resources of smart scale 100, a client device of a user connected and communicating with smart scale 100, and/or remote computing system(s) (e.g., servers, such as cloud-based servers). In such cases, controller system 102 may rely on the more powerful processing capabilities and larger datasets of historical health metric(s) stored on the client device and/or remote computing system(s) to determine the recommendations.

In some cases, a user interface (UI) associated with smart scale 100 may enable users, such as users 116, to customize the color scheme or color-coded feedback provided by controller system 102 of smart scale 100. In some instances, the UI may be displayed on a client device (e.g., on a display device of the client device) of a user, such as user 116. In some aspects, the UI may be displayed by smart scale 100 (e.g., on a display device of smart scale 100). In some instances, the UI may include one or more interface elements (e.g., toggles, sliders, buttons, drop-down menus, checkboxes, etc.) that enable a user to select a type of health metric (e.g., weight, body fat (e.g., percentage), body mass index (BMI), skeletal muscle (e.g., percentage), fat-free mass (e.g., lbs., oz, g, etc.), subcutaneous fat (e.g., percentage), visceral fat, body water (e.g., percentage), muscle mass e.g., lbs., oz, g, etc.), bone mass (e.g., lbs., oz, g, etc.), protein (e.g., percentage), basal metabolic rate (BMR), metabolic age (e.g., years))controller system 102 may cause display system 106 to output colors for. Based on the selected type of health metric and color map data, controller system 102 and/or the client device may identify a color map associated with the health metric. Based on the color map, controller system 102 may cause display system 106 to output colors corresponding to the type of health metric the user selected.

For instance, a user may select, via the UI, body fat percentage as the type of health metric to output corresponding colors to. Based on the selection, controller system 102 and/or the client device may identify a color map associated with body fat percentage. Based on a determined health metric of the user and the identified color map, controller system 102 may perform any of the processes described herein to out a corresponding color (e.g., controller system 102 may cause display system 106 to output the color yellow indicating the determined health metric associated with body fat percentage is high).

In some aspects, the interface element(s) of the UI may enable a user to customize the color scheme or color-coded feedback associated with a particular health metric value or a range of health metric values. A user may select, via the interface element(s) a color map associated with a health metric type, such as body fat percentage. The user may adjust a color associated with one or more health metrics or ranges of health metrics via the interface element(s). Based on a determined health metric of the user, controller system 102 may use the adjusted or customized color map to out a corresponding color in accordance with the customized or adjusted color map.

For instance, a user may select, via the interface element(s), a color map for body fat percentage. The user may adjust one or more colors of the color map. Each of the colors may be associated with a body fat percentage or a range of body fat percentages. Based on a determined body fat percentage of the user and the color map, controller system 102 may perform any of the processes described herein to out a corresponding color associated with the determined fat percentage (e.g., the adjusted or customized color may be blue).

In some examples, reminder system 110 may prompt or remind a user, such as user 116, to weigh in or measure themselves using smart scale 100. Reminder system 110 may generate reminders or prompts for the user, enhancing engagement and helping maintain consistent health measurements. In some aspects, reminder system 110 may communicate with display system 106 to output the reminder. The output may include a variety of visual indicators, such as color scheme or color-coded feedback, text messages, images, or videos, as well as audio outputs (e.g., sounds or spoken messages) to notify the user.

In some aspects, reminder system 110 may provide a prompt or reminder at a predetermined time. The predetermined time may be set by the user through a UI associated with smart scale 100 (e.g., via one or more interface elements that enable the user to set the predetermined time). For example, user 116 may configure the predetermined time via the UI. User 116 may configure the predetermined time as 7:00 AM. Based on the configuration, reminder system 110 may cause display system 106 to output a reminder for user 116 to weigh in at 7:00 AM (e.g., display device of display system 106 may illuminate and show a visual message, such as “Time to weigh in!” and/or the light source of display system 106 may cause a platform of measurement system 104 to illuminate a particular color (e.g., also set/configured via the UI)). In some instances, the UI may be displayed on a display device of display system 106. Additionally, or alternatively, the UI may be displayed on a client device communicating with smart scale 100.

In some aspects, the predetermined time may be determined by reminder system 110 based on behavioral data of the user. For example, reminder system 110 may access profile information (e.g., a user profile) of a user to identify each time the user has weighed or measured themselves using smart scale 100 and corresponding timestamps. Based on the profile information, reminder system 110 may determine the likely time the user typically uses smart scale 100. Reminder system 110 may determine such determined time is the predetermined time for the reminder.

In some cases, reminder system 110 provide the reminder or prompt based on whether a user (e.g., user 116) is detected in the vicinity of smart scale 100 (e.g., within a proximity distance threshold to smart scale 100). Based on the detecting the user is in the vicinity of smart scale 100, reminder system 110 may cause display system 106 to output or provide a prompt or reminder for the user.

In some instances, reminder system 110 may use one or more sensors integrated with smart scale 100, such as a microphone that may be included with measurement system 104 to detect sound changes indicative of user movement and/or various types of proximity sensors, including infrared (IR) sensors, ultrasonic sensors, passive infrared (PIR) sensors, and radar sensors. Such sensor(s) may detect the approach of a user based on changes in environmental data or physical movement. Additionally, or alternatively, reminder system 110 may employ location-based technologies (e.g., Global Positioning System (GPS), Wi-Fi, Bluetooth, or Near Field Communication (NFC), etc.) to determine the proximity of a client device of a user (e.g., smartphone, smartwatch). By analyzing the signal strength or positional data from the client device, reminder system 110 can identify when the user is approaching or is near smart scale 100 (e.g., within a proximity distance threshold). Based on the user approaching or is near smart scale 100, reminder system 110 may cause display system 106 to output a reminder or prompt. For example, if user 116 is detected nearby based on proximity sensors or Bluetooth signals from the user's smartphone, reminder system 110 may cause display system 106 to output a reminder for user 116 (e.g., display device of display system 106 may illuminate and show a visual message, such as “Time to weigh in!” and/or the light source of display system 106 may cause a platform of measurement system 104 to illuminate a particular color (e.g., also set/configured via the UI)).

In some aspects, reminder system 110 may function while one or more other functions or systems (e.g., controller system 102, measurement system 104, display system 106, etc.) of smart scale 100 is in a low-power mode or low-power state (e.g., reduced functionality to conserve battery life). During low-power mode, reminder system 110 may activate display system 106 to output the reminder or prompt for the user (e.g., user 116) to weigh in or conduct a health measurement session. As described herein, reminder system 110 may cause display system 106 to output the reminder or prompt at a predetermined time or when a user is near smart scale 100 and/or within a predetermined proximity threshold to smart scale 100.

In some examples, low-light system 112 may provide an environment with low light conditions with low-level, eye-friendly lighting when a user (e.g., user 116) is detected within a proximity distance threshold to smart scale 100. In some aspects, low-light system 112 may determine whether smart scale 100 is located in an environment with low light conditions. Based on smart scale 100 being in an environment with low light conditions, low-light system 112 may determine when a user is within a proximity distance threshold to smart scale 100. Based on detecting the user is within the proximity distance threshold to smart scale 100, low-light system 112 may communicate with display system 106 to output a low intensity light, ranging from approximately 0.1 lux to 100 lux to illuminate the environment with the low light conditions.

In some cases, low-light system 112 may process sensor data of one or more sensors determine whether smart scale 100 is located in an environment with low light conditions. For example, smart scale 100 may include one or more photodetectors that generate sensor data indicating an intensity of light level in an environment smart scale 100 is in (e.g., a lux value representing or corresponding to the intensity of light in the environment). Low-light system 112 may obtain the sensor data and compare the indicated intensity of light level to a predetermined criteria threshold (e.g., a lux value representing a low-light threshold). Based on the comparison, low-light system 112 may determine whether smart scale is in an environment with a low light conditions (e.g., a lux value representing the intensity of light in the environment is equal to or lower than the lux value representing a low-light threshold). Additionally, or alternatively, low-light system 112 may access time-of-day data from a paired client device (e.g., smartphone) via wireless communication to determine whether the ambient light conditions are likely to be low, such as during nighttime hours.

In some instances, low-light system 112 may use one or more sensors to detect whether a user is within a proximity distance threshold to smart scale 100. Examples of the sensor(s) include, but are not limited to, a microphone and proximity sensors (e.g., IR sensors, ultrasonic sensors, passive infrared (PIR) sensors, radar sensors, etc.). Additionally, or alternatively, low-light system 112 may use location-based technologies (e.g., Global Positioning System (GPS), Wi-Fi, Bluetooth, near field communication (NFC), etc.) to detect when the user or a client device of the user is within a proximity distance threshold to smart scale 100. Based on detecting the user is within the proximity distance threshold to smart scale 100, low-light system 112 may cause display system 106 output a low-level, eye friendly lighting.

In some aspects, the low-level, eye friendly lighting may be a light in a low-level intensity range with an output power between 0.01-1.0 watts. The described power range may cause the light to be gentle, minimizing retinal stimulation and reducing the risk of eye strain or potential harm to the eyes of the user. In some examples, the low-level, eye friendly lighting may be a light in a low-level intensity range with a wavelength range of approximately 500 to 600 nanometers, corresponding to warm colors, such as yellow, amber, or soft orange. The warm colors may be less likely to interfere with melatonin production or disrupt the user's sleep cycle, compared to cooler, blue-spectrum light (below 480 nanometers). In some cases, the warm colored, low-light intensity light output may enable the eyes of a user near the smart scale in a low-light condition to quickly adapt to the low-light conditions without causing shock or discomfort. In some examples, the warm light provides sufficient illumination for the user to safely navigate a location in a low light level condition, reducing the risk of accidents while ensuring a non-intrusive experience that does not disturb the user's night vision or sleep.

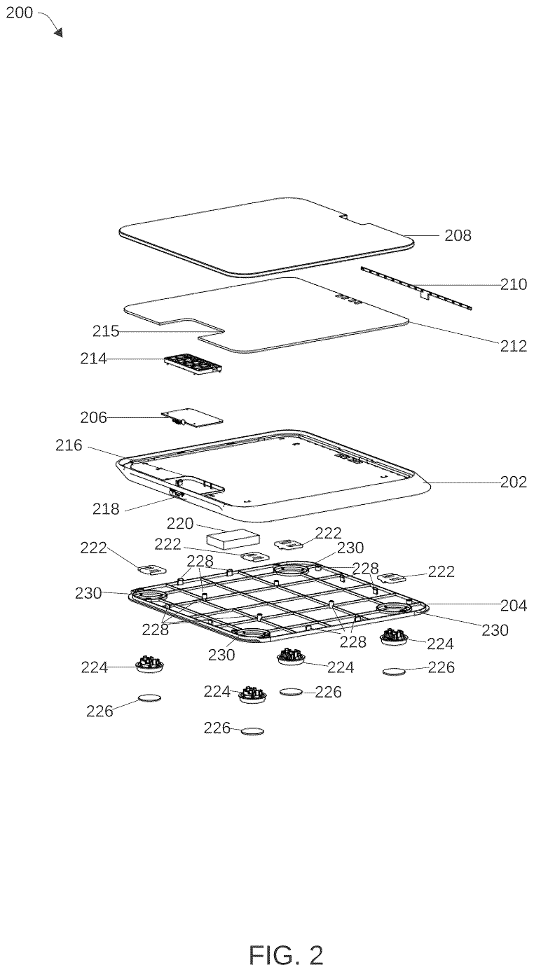

FIG. 2 illustrates an exploded view of one or more components that may be included in a smart scale, such as smart scale 100 of FIG. 1. As illustrated in FIG. 2, smart scale 200 may include one or more housing elements. The housing element(s) may include top cabinet 202 and bottom cabinet 204. In some examples, the housing element(s) may provide structural support for component(s) of smart scale 200. Examples of components of smart scale 200 top cabinet 202 may provide structural support for, include, but are not limited to, control device 206, platform 208, light source device 210, backlight board 212, display device 214, battery 220, one or more sensors 222, one or more support elements 224 and/or one or more grip elements 226. In some instances, the housing element(s) may include one or more support elements 228. As illustrated in FIG. 2, support element(s) 228 (e.g., support column(s)) may prevent top cabinet 202 from sagging or bending under pressure when a user is on platform 208.

In some cases, control device 206 may implement or perform any of the example processes described herein, such as, but not limited to, determining health metric(s) for users, providing the determined health metric(s) to the users (e.g., via a UI associated with smart scale 200), including, but not limited to, health metric(s) control device 206 determined, health metrics that reflect the users'progress over time, and/or patterns and predictions about the users'health and wellness journey, providing, to the users a color scheme or color-coded feedback corresponding to the health metric(s) associated with the users, enabling users to customize color maps (e.g., via UI associated with smart scale 200), enabling users to create and configure user profiles with smart scale 200 (e.g., via UI associated with smart scale 200), enabling users to manually select corresponding profiles control device 206 may associate determined health metric(s) to (e.g., via UI associated with smart scale 200), automatically selecting user profiles for users that control device 206 may associate determined corresponding health metric(s) to, providing prompts or reminders for users to weigh in or measure themselves using smart scale 100, determining behavior data or information, enabling users to set a time for the reminder(s/prompt(s), providing a low-level, eye friendly illumination (e.g., 0.1 lux to 100 lux) for locations with low light conditions the users and smart scale 100 are in, determining characteristics of the users standing on smart scale 200, identifying users that may be using or may be about to use smart scale 200, detecting when a user is near smart scale 200 or within a proximity distance threshold to smart scale 200, as similarly described with controller system 102, profile management system 108, reminder system 110, and/or low-light system 112 of FIG. 1. Control device 206 may include one or more processors to implement or perform any of the example processes described herein. In some instances, control device 206 may include a memory that may store, color map data, data characterizing and identifying health metric(s) of one or more users of smart scale 100 and corresponding timestamps, profile information for the user(s), and/or health and wellness device data. In some aspects, control device 206 may be electrically coupled to light source device 210, backlight board 212, display device 214, battery 220, one or more sensors 222, and/or in some instances, one or more electrodes included in platform 208.

In some instances, top cabinet 202 may include one or more structural elements, such as structural element(s) 216 that provide structural support for control device 206. As illustrated in FIG. 2, structural element(s) 216 may provide a compartment within top cabinet 202 for control device 206. In some example, control device 206 may include a microphone. As described herein, the microphone may enable control device 206 to determine whether a user is near or within a proximity distance threshold to smart scale 200 (e.g., detect sound changes indicative of user movement. In some cases, control device 206 may include a power interface (e.g., USB type A, USB type B, USB type-C, micro-USB, mini USB, etc.) that enables control device 206 to charge or recharge battery 220, or provide power to smart scale 200 (e.g., the electrical components of smart scale 200). In such cases, structural element(s) 216 may include a channel, port or opening that enables a power cable to provide power to battery 220 and/or to smart scale 200 via the power interface. Examples of batteries for battery 220 include, rechargeable batteries (e.g., lithium-ion batteries, lithium polymer batteries, nickel-metal hydride batteries, nickel cadmium batteries, lead-acid batteries, etc.) or non-rechargeable batteries (e.g., alkaline batteries, lithium batteries, zinc-carbon batteries, etc.). Battery 220 may provide power to one or more electrical components of smart scale 200, such as control device 206, light source device 210, backlight board 212, display device 214, one or more sensors 222, and/or in some instances, one or more electrodes included in platform 208.

In some example, platform 208, similar to the platform of measurement system 104, may include one or more electrodes (e.g., embedded in platform 208). As described herein, platform 208 may be formed out of a conductive and/or transparent material (e.g., indium tin oxide). Additionally, or alternatively, platform 208 may be formed out of a material with high mechanical strength to at least withstand a weight of a user standing on the platform (e.g., approximately at most 500 pounds or 226 kilograms). In such examples, control device 206 may include one or more electrical current generators. The electrical current generator(s) may be electrically coupled to the electrode(s) and may provide that provide an electrical signal to each of the electrode(s). The electrical signal may pass through a body of a user standing on platform 208. The electrode(s) may receive the electrical signal from the body of the user. Control device 206 may receive the electrical signal from the body of the user via the electrode(s). As described herein, control device 206 may determine one or more health metrics of the user based in part on the electrical signal (e.g., via BIA). In some instances, platform 208 may include an insulator that may divide platform 208 into multiple regions.

For instance, platform 208 may include an insulator that divides platform 208 into four regions. Each of the four regions may include an electrode embedded into the corresponding region of platform 208. When a user stands on platform 208, their left foot may be positioned on two of the regions, such as the first region and the third region, while their right foot may be positioned on the other two regions, such as the second region and the fourth region. Corresponding electrical current generator(s) may provide a sinusoidal electrical signal to the electrodes in the two regions associated with the user's left foot (e.g., the first region and the third region). The sinusoidal electrical signal passes through the left foot, left leg, right leg, and right foot, and is then received and detected by the electrodes of the regions associated with the right foot (e.g., The second region and the fourth region). The electrical signal received from the electrodes in the regions associated with the right foot may provide or transmitted to control device 206. In some instances, the electrical received from the electrodes in the regions associated with the right foot may be amplified, rectified and/or undergo analog-to-digital conversion (A/D conversion) by one or more electrical components of control device 206 (e.g., amplifier, rectifier, and A/D converter, respectively) prior to being received by control device 206

In some examples, light source device 210, similar to a light source of display system 106 of FIG. 1, may provide or emit light corresponding to heath metric(s) of a user. In some cases, light source device 210 may output or emit a light in accordance with parameters or instructions of control device 206, such as color and brightness. In some instances, light source device 210 may include one or more LEDs (e.g., an array of LEDs, such as a light strip). In such instances, control device 206 may independently adjust or configure parameters of each of LED(s) of light source device 210, such as the brightness and/or color of each of the LED(s) (e.g., RGB parameters). As described herein, the color and/or brightness of the light emitted by light source device 210 may be based on health metric(s) of a user and corresponding color map(s).

For example, when a user steps onto platform 208, control device 206 may determine real-health metric(s) based at least on electrical signals received from one or more electrodes of platform 208 and/or sensor data obtained from the one or more sensors 222. Based on corresponding color map(s) of color map data, control device 206 may adjust or define the output of the RGB LEDs accordingly. For instance, if the detected BMI value is within a predetermined range associated with a user being overweight, control device 206 may cause the LEDs to emit a red colored light.

In some cases, backlight board 212, similar to a backlight board of display system 106 of FIG. 1, may be configured to ensure even illumination of the light emitted by light source device 210. In some instances, backlight board 212 may include a reflection board, light guide plate, reflective films, light diffusing film and/or, in some instances a protective plate. As described herein, the reflection board redirects light emitted from light source device 210 upward enhancing the efficiency of light utilization, the light guide plate is configured to distribute the light emitted from light source device 210 uniformly across a surface, such as a surface formed out of transparent, the reflective film(s) may enhance light distribution and minimize light leakage from light source device 210, the light diffusing film scatters the light emitted from light source device 210 evenly, reducing hotspots and ensuring that the color output is consistent across the entire display area, and the protective plate may protect the other components of the backlight board while maintaining optical clarity.

As illustrated, light source device 210 and/or backlight board 212 may be coupled to platform 208. In examples where platform 208 is formed out of a transparent or translucent material, such as a conductive glass or acrylic material, light source device 210 and/or backlight board 212 may emit colored light to illuminate the one or more portions of the surface of platform 208. In some instances, light source device 210 and/or backlight board 212 may be positioned underneath platform 208 (e.g., the reflection board may be positioned at the base of backlight board 212 and reflects the light emitted from light source device 210 towards platform 208, the light guide path is positioned above the reflection board, the reflective films may be layered above the light guide plate, the light diffusing films may be positioned above the reflective films, and the protective plate is positioned above backlight board 212).

In some cases, backlight board 212 may enable control device 206 to provide light of two or more different colors at once on platform 208. Each of the two or more colors may correspond to a health metric of a user control device 206 may have determined. In such cases, platform 208 may include two or more zones or areas. Backlight board 212 may include components that divide or segment the platform into the two or more zones or areas. Examples of such components, include but are not limited to, light guide plate configured with microstructures that direct light emitted from light source device 210 even across the surface to prevent color mixing or uneven illumination, two or more segmented or zoned diffusing films that may enable and maintain color differentiation between adjacent areas allowing the platform to display multiple colors simultaneously without the colors mixing or blending, and/or two or more optical barriers or dividers that separate the zones, prevent potential color overlap between each zone, and maintain sharp color boundaries between the zones. Controller system 102 may cause light source device 210 to output different colors for each of the two or more zones in accordance with corresponding health metrics and associated color maps.

In some aspects, light source device 210 and/or backlight board 212, as similarly described with light source of display system 106 and/or backlight board of display system 106 may emit a light corresponding to a reminder, and/or for environments with low-light conditions when users are nearby or within a proximity distance threshold. In such aspects, control device 206 may cause light source device 210 and/or backlight board 212 may emit a light corresponding to a reminder, and/or for environments with low-light conditions when users are nearby or within a proximity distance threshold, as similarly described with reminder system 110 and low-light system 112, respectively.

In some examples, display device 214, similar to a display device of display system 106, may be configured to output numerical measurement(s) associated with heath metric(s) determined by control device 206, information of the patterns and predictions about the user's health and wellness journey determined by control device 206, recommendations, and/or reminders. In some cases, control device 206 may provide the heath metric(s), the information of patterns and predictions about the user's health and wellness journey, recommendations and/or reminds to display device 214 to cause display device 214 to output the corresponding numerical measurement(s) associated with heath metric(s) determined by control device 206, information of the patterns and predictions about the user's health and wellness journey determined by control device 206, recommendations, and/or reminders, respectively, as similarly described with controller system 102, the display device of display system 106, reminder system 110, and low-light system 112. In some instances, display device 214, similar to a display device of display system 106, may enable users to create, configure and manage individual user profiles. In such instances, control device 206 may provide a UI associated with smart scale 200, as similarly described with profile management system 108.

In some instances, display device 214 may be positioned under platform 208. In some cases, display device 214 may be positioned over control device 206. In some examples, backlight board 212 may be dimensioned to fit display device 214. For instance, and as illustrated in FIG. 2, backlight board 212 may include recess 215. Recess 215 may be dimensioned such that display device 214 may fit in recess 215. Recess 215 may prevent backlight board 212 from obstructing the view of display device 214.

In some aspects, sensor(s) 222 (e.g., pressure sensor(s)), as similarly described with the sensor(s) of measurement system 104, may be configured to generate sensor data indicating a weight of a user. As described similarly with controller system 102, control device 206 may determine a weight of a user standing on platform 208 based on the sensor data generated by sensor(s) 222. Additionally, or alternatively, control device 206, as similarly described with controller system 102, may determine one or more other heath metrics (e.g.,, body fat (e.g., percentage), body mass index (BMI), skeletal muscle (e.g., percentage), fat-free mass (e.g., lbs., oz, g, etc.), subcutaneous fat (e.g., percentage), visceral fat, body water (e.g., percentage), muscle mass e.g., lbs., oz, g, etc.), bone mass (e.g., lbs., oz, g, etc.), protein (e.g., percentage), basal metabolic rate (BMR), metabolic age (e.g., years) based in part on the sensor data.

As described herein, smart scale 200 may include one or more other sensors (not illustrated in FIG. 2). The other sensors may include proximity sensor(s) (e.g., infrared (IR) sensors, ultrasonic sensors, passive infrared (PIR) sensors, radar sensors, etc.) and/or photodetector(s). In some instances, and as similarly described with reminder system 110 and/or low-light system 112, control device 2016 may use the proximity sensor(s) to determine a location of a user and/or a client device of the user. Based on the location of the user and/or the client device of the user, control device 206 may determine whether the user is within a proximity distance threshold of smart scale 200 or is near smart scale 200. In some examples, and as similarly described with low-light system 112, control device 2016 may use the proximity sensor(s) to determine whether smart scale 200 is located in an environment with low light conditions.

In some cases, each of sensor(s) 222 may be positioned below platform 208. In some instances, each of sensor(s) 222 may be coupled to a corresponding support element 224 (e.g., via latch or coupling mechanism). Support element 224 may prevent a corresponding sensor 222 from moving and thereby increasing the accuracy of weight measurement. Support element 224 may be cylindrical, rectangular or polygonal. Support element 224 may be coupled or attached to bottom cabinet 204.

In some examples, bottom cabinet 204 may include provide additional structural support for each of support element 224 to provide additional support and stability for support element 224 and corresponding sensor 222. For example, and as illustrated in FIG. 2, bottom cabinet 204 may include one or more cavities, openings or holes, such as cavity 230, dimensioned to accommodate each support element 224 for additional support and stability. Cavity 230 may be dimensioned to match the shape of support element 224, such as a cylindrical shape, a rectangular shape or polygonal shape, enabling a close fit with corresponding support element 224. In some instances, a close fit may be defined as a close tolerance fit between cavity 230 and corresponding support element 224 (e.g., a dimensional variation of not more than ±0.1 mm, ensuring that corresponding support element 224 fits snugly without gaps or excessive force within cavity 230). In some cases, a close fit may be defined as a friction fit between cavity 230 and corresponding support element 224 (e.g., cavity 230 is configured to have dimensions that create sufficient frictional resistance when corresponding support element 224 is inserted into cavity 230, preventing unintended movement or dislodgment). One or more cavities 230, be positioned at a corner of bottom cabinet 204 (e.g., cavity 230 at each corner of bottom cabinet 204). In some aspects, support element 224 may couple to a corresponding cavity 230 using one or more fittings or mechanisms (e.g., friction fit, threaded fit, adhesive bonding, welded connection, snap fit, pin and lock mechanism, magnetic coupling, tapered fit, etc.).

In some instances, each support element 224 may include or be coupled to a corresponding grip element 226. Grip element 226 may be formed out of a high friction material (e.g., a material that generates a significant resistance to sliding or movement when in contact with another surface), such as rubber. Grip element 226 may prevent smart scale 200 from moving, sliding, and/or shifting when a user steps up on and/or stands on smart scale 200.

In some cases, controller system 102 may provide to a user, such as user 116, a recommendation including personalized feedback for the user during their health and wellness journey based on health metric(s) determined by controller system 102. In some examples, the recommendation can also identify one or more health and wellness device(s) the user may use for their health and wellness journey. Examples of health and wellness device(s) the recommendation may identify include health and wellness devices included in a health and wellness environment of smart scale 100, such as, but not limited to a bicycle, a jump rope, a smart food scale, a blood pressure monitor, etc.

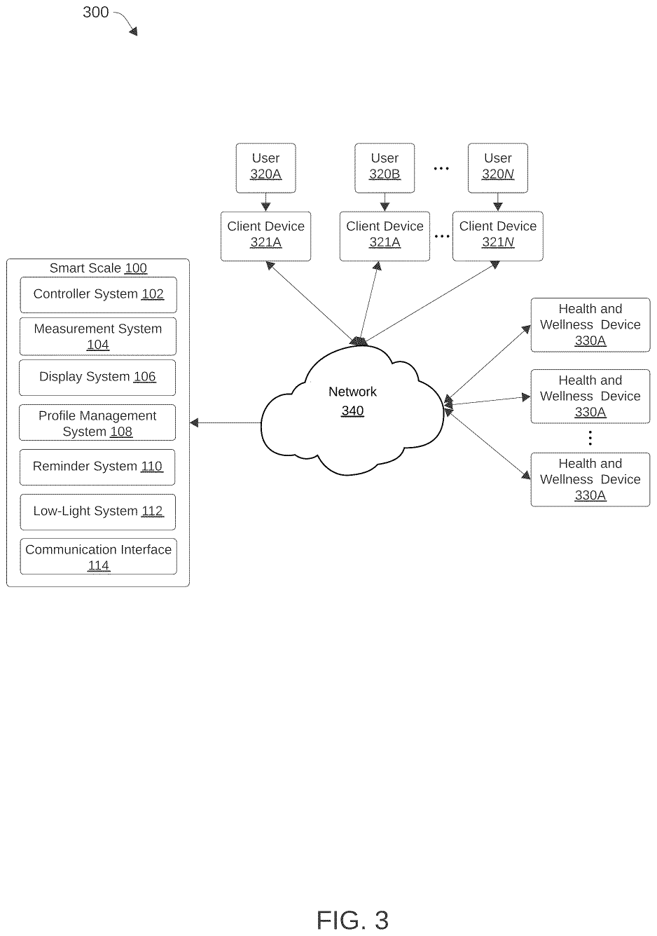

FIG. 3 illustrates a block diagram of an example health and wellness environment 300, according to some embodiments. Health and wellness environment 300 may include smart scale 100, one or more client devices 321 (e.g., client device 321A, client device 321B... client device 321N) operated by a corresponding user 116 (e.g., user 116A, user 116B . . . user 116N) and one or more health and wellness devices 330 (e.g., health and wellness device 330A, health and wellness device 330B . . . health and wellness device 330N). As illustrated in FIG. 3, smart scale 100 may communicate with client device(s) 321, and/or health and wellness device(s) 330 over network 340 and via communication interface 114 (e.g., a cable modem, a satellite television (TV) transceiver, a router, an access point, a network interface card, an antenna, etc.). Network 340 can include one or more public and/or private networks. In some examples, the network 145 can include, without limitation, the Internet; a wide area network (WAN); a backbone network; a cloud network; a local area network (LAN); a datacenter network; a network segment; an Internet Service Provider (ISP) network; a wireless LAN; an intranet; an extranet; a wired and/or wireless network such as a cellular network, a Bluetooth network or link, an infrared network or link, a WIFI network, etc. ; and/or any other short range, long range, local, regional, global communications mechanism, means, approach, protocol and/or network, or any combination(s) thereof. Smart scale 100, such as controller system 102, may provide to one or more users 116, health and wellness related recommendations including information associated with health and wellness device(s) 330 users 116 may use for their health and wellness journey.

In some cases, controller system 102 may process health and wellness device data stored in one or more database storage systems (e.g., a memory of smart scale 100, a memory or storage device of at least one of the client devices 321, and/or one or more remote computing systems or databases) to identity the health and wellness device(s) 330 that may be included in the recommendations. The health and wellness device data may include information such as, but not limited to, for each health and wellness device 330 within health and wellness environment 300, corresponding device identifier (e.g., unique identifiers, such as MAC addresses, and/or serial numbers, for identifying the corresponding health and wellness device 330 within health and wellness environment 300), device capabilities (e.g., information about the functions the corresponding health and wellness device 330 supports, such as the type of exercise the corresponding health and wellness device 330 supports, and other functions, such as calorie tracking, heart rate monitoring, exercise tracking, etc.), device associations (e.g., links between corresponding health and wellness device 330 and specific users 116), and communication protocols (e.g., supported wireless protocols, such as, Bluetooth and/or Wi-Fi, for compatibility with smart scale 100). Controller system 102 may determine, for a recommendation for a particular user 116, health and wellness device(s) 330 within health and wellness environment 300 that are available for the user 116 and related to the corresponding recommendation (e.g., the health metrics the recommendation is based on).

For example, user 116 may step onto the platform of measurement system 104. Controller system 102 may determine multiple health metrics associated with the user, including a BMI value, a muscle mass percentage and a body fat percentage based on electrical signals and/or sensor data generated by measurement system 104. Controller system 102 may determine one or more health and wellness devices 330 within health and wellness environment 300 that are available to user 116 and are related to the determined health metrics based on health and wellness device data stored in a memory of smart scale 100 (not illustrated). Based on the health metrics of user 116 and the determined health and wellness device(s) 330, controller system 102 may generate a recommendation for user 116. The recommendation may provide a personalized feedback for a health and wellness journey of user 116 and may identify associated health and wellness device(s) 330 that user 116 may use for the health and wellness journey of user 116. For instance, based on the health metrics of the user indicating the user has a high BMI value, a low muscle mass percentage and a high body fat percentage, controller system 102 may determine a recommendation suggesting the user may increase aerobic exercise and/or monitor their caloric intake to at least reduce body fat levels. Based on the health and wellness device data, controller system 102 may determine health and wellness device 330B (e.g., a stationary bike) is available for use (e.g., may be communicating with smart scale 100) by user 116 and may enable user 116 to increase their aerobic exercise.

In some instances, controller system 102 may communicate with health and wellness device 330 identified in a recommendation to a user, such as user 116, that the user subsequently uses after the recommendation is provided to the user. Controller system 102 may to track and monitor the progress of the user based on the communications. Data generated from tracking and monitoring the progress of the user may be used to provide a more comprehensive health and wellness diagnostics for determining the progress of the health and wellness journey of the user.

In some instances, if the health and wellness device data indicates there are no available health and wellness devices 330 (e.g., no suitable health and wellness device 330, or no health and wellness device 330 that is related to the health metrics of the user, or no health and wellness device 330 within health and wellness environment 300), such as user 116, controller system 102 may provide options to the user to acquire suitable health and wellness device 330. In some examples, controller system 102 may provide the options by providing a UI associated with smart scale 100. In some instances, the UI may provide content associated with the options, such as an advertisement for the suitable health wellness device 330. The UI may include one or more interface elements that enable the user to obtain health and wellness device 330. In some cases, the UI may be displayed on a display device of display system 106. In some aspects, the UI may be displayed by a client device of the user.

For example, user 116 may step onto the platform of measurement system 104. Controller system 102 may determine health metrics indicating user 116 has a high BMI value, a low muscle mass percentage and a high body fat percentage based on electrical signals and/or sensor data generated by measurement system 104. Controller system 102 may determine there are no available health and wellness devices 330 within health and wellness environment 300 for user 116 based on health and wellness device data stored in a remote database accessed by controller system 102 (not illustrated). Based on determining there are no available health and wellness devices 330 for user 116, controller system 102 may provide a UI associated with smart scale 100 to user 116. The UI may include interface element(s) that inform user 116 of options to obtain health and wellness device 330. The interface element(s) of the UI may enable user 116 to obtain health and wellness device 330 in accordance with the identified options. For instance, the interface element(s) may provide a recommendation based on the health metrics to user 116 suggesting user 116 may increase aerobic exercise and/or monitor their caloric intake to at least reduce body fat levels. Further, the interface element(s) may recommend using health and wellness device 330A, such as a stationary bike or jump rope, to help increase user 116's aerobic exercise. The interface element(s) may enable user 116 to obtain health and wellness device 330A, and in some instances may provide options for obtaining health and wellness device 330A.

In some examples, the health and wellness data may indicate whether client device 321 of user 116 is a fitness tracker or health and wellness tracker (e.g., a smartwatch or fitness band). In such examples, controller system 102 may obtain one or more health metrics determined and/or generated by the fitness tracker (e.g., heart rate, activity levels, caloric expenditure, etc.). Controller system 102 may integrate the health metric(s) associated with the fitness tracker with the health metric(s) determined by controller system 102. The integrated health metrics may indicate a more comprehensive feedback to the health and wellness of a user.

For example, if controller system 102 determines a health metric indicates an increase in a weight of user 116 but the health metric associated with the fitness tracker indicates high physical activity and a low resting heart rate, controller system 102 may determine that the weight gain is due to increased muscle mass. In some instances, controller system 102 may enable display system 106 and/or a client device to display an output corresponding to the integrated health metrics, such as following the example above, “your weight increase appears to be due to muscle gain, as your fitness tracker indicates high activity levels and excellent cardiovascular health—good job!”

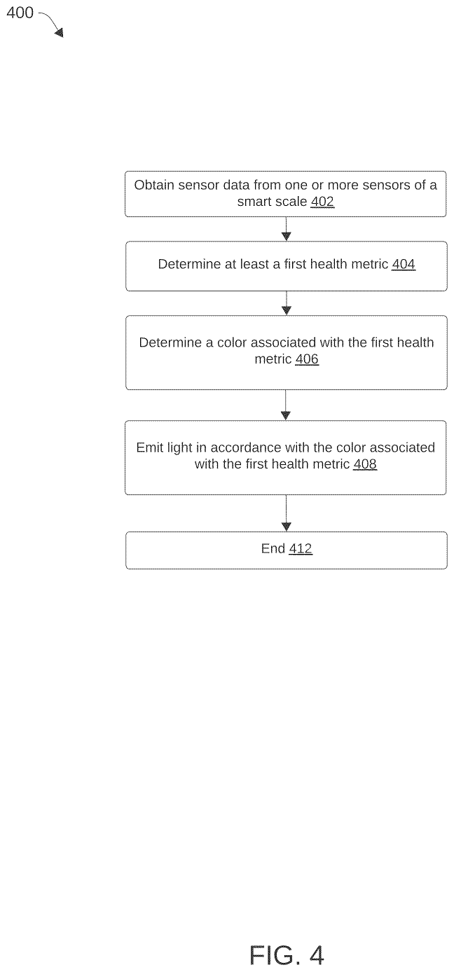

FIG. 4 is a flowchart for a method 400 for enabling an example smart scale to output colors corresponding to health status(es) of a user, according to some examples of the present disclosure. Method 400 can be performed by processing logic that can comprise hardware (e.g., circuitry, dedicated logic, programmable logic, microcode, etc.), software (e.g., instructions executing on a processing device), or a combination thereof. It is to be appreciated that not all steps may be needed to perform the disclosure provided herein. Further, some of the steps may be performed simultaneously, or in a different order than shown in FIG. 4, as will be understood by a person of ordinary skill in the art.

Method 400 shall be described with reference to FIGS. 1-2. However, method 400 is not limited to those examples. In step 402, controller system 102 may obtain sensor data from one or more sensors of smart scale 100. As described herein, smart scale 100 may include one or more sensors, such as a pressure sensor. The one or more sensors may generate sensor data that indicates a health metric, such as a weight of the user.

In step 404, controller system 102 may determine at least a first health metric. In some examples, controller system 102 may determine the first health metric based at least one the sensor data. In some cases, controller system may obtain an electrical signal from one or more electrodes of smart scale 100. In such cases, controller system may determine the first health metric based on the sensor data and/or the electrical signal. Examples of health metric(s) controller system 102 may determine based on the sensor data and the electrical signal include, but are not limited to, body fat (e.g., percentage), body mass index (BMI), skeletal muscle (e.g., percentage), fat-free mass (e.g., lbs., oz, g, etc.), subcutaneous fat (e.g., percentage), visceral fat, body water (e.g., percentage), muscle mass e.g., lbs., oz, g, etc.), bone mass (e.g., lbs., oz, g, etc.), protein (e.g., percentage), basal metabolic rate (BMR), and metabolic age (e.g., years). In some instances, controller system 102 may obtain electrical signals and/or sensor data generated by electrode(s) and/or sensor(s) of smart scale 100 while the user is standing a platform of smart scale 100. The electrode(s) may be included or embedded in the platform. The sensor(s) may be coupled to the platform to generate sensor data indicating a weight of the user.

In step 406, controller system 102 may determine a color associated with the first health metric. In some examples, controller system 102 may determine a color associated with the first health metric based on color map data associated with the first health metric. In some cases, controller system 102 may identify a color map associated with the type of health metric controller system 102 determined, such as the first health metric. The color map may identify one or more colors and corresponding health metric value or range of health metric values. Based on the color map, controller system 102 may identify a range of health metric values a value of the first health metric is associated with (e.g., falls within a range of health metric values) and corresponding color. Controller system 102 may determine the color associated with the range of health metric values the first health metric is associated with, is the color associated with the first health metric. Such color may be outputted by controller system 102.

In step 408, controller system 102 may emit a light, via display system 106, in accordance with the color associated with the first health metric. In some examples, the light being emitted towards one or more portions of a platform of the smart scale and causing the platform to emit light in the color associated with the first health metric. In some cases, display system 106 may include a light source, such as one or more LEDs, and, in some instances, a backlight board. In such cases, controller system 102 may cause the light source to emit light in accordance with the determined color. In some aspects, the light source, and in some instances, the backlight board may be coupled to a translucent or transparent platform of smart scale 100. In such aspects, the light emitted from the light source, and in some instances, the backlight board, may illuminate one or more portions of the platform. The light illuminating or emitting from the platform may be in the color associated with the first health metric.

### Example Neural Network Architectures

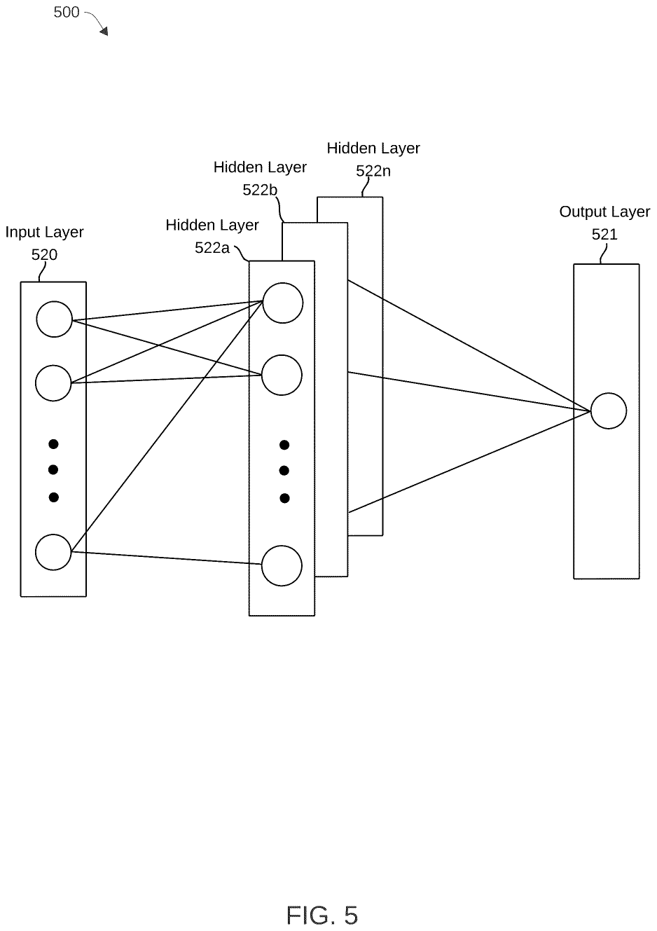

FIG. 5 is a diagram illustrating an example of a neural network architecture 500 that can be used to implement some or all of the neural networks described herein. The neural network architecture 500 can include an input layer 520 that can be configured to receive and process data to generate one or more outputs. The neural network architecture 500 also includes hidden layers 522a, 522b, through 522n. The hidden layers 522a, 522b, through 522n include “n” number of hidden layers, where “n” is an integer greater than or equal to one. The number of hidden layers can be made to include as many layers as needed for the given application. The neural network architecture 500 further includes an output layer 521 that provides an output resulting from the processing performed by the hidden layers 522a, 522b, through 522n.

The neural network architecture 500 is a multi-layer neural network of interconnected nodes. Each node can represent a piece of information. Information associated with the nodes is shared among the different layers and each layer retains information as information is processed. In some cases, the neural network architecture 500 can include a feed-forward network, in which case there are no feedback connections where outputs of the network are fed back into itself. In some cases, the neural network architecture 500 can include a recurrent neural network, which can have loops that allow information to be carried across nodes while reading in input.

Information can be exchanged between nodes through node-to-node interconnections between the various layers. Nodes of the input layer 520 can activate a set of nodes in the first hidden layer 522a. For example, as shown, each of the input nodes of the input layer 520 is connected to each of the nodes of the first hidden layer 522a. The nodes of the first hidden layer 522a can transform the information of each input node by applying activation functions to the input node information. The information derived from the transformation can then be passed to and can activate the nodes of the next hidden layer 522b, which can perform their own designated functions. Example functions include convolutional, up-sampling, data transformation, and/or any other suitable functions. The output of the hidden layer 522b can then activate nodes of the next hidden layer, and so on. The output of the last hidden layer 522n can activate one or more nodes of the output layer 521, at which an output is provided. In some cases, while nodes in the neural network architecture 500 are shown as having multiple output lines, a node can have a single output and all lines shown as being output from a node represent the same output value.

In some cases, each node or interconnection between nodes can have a weight that is a set of parameters derived from the training of the neural network architecture 500. Once the neural network architecture 500 is trained, it can be referred to as a trained neural network, which can be used to generate one or more outputs. For example, an interconnection between nodes can represent a piece of information learned about the interconnected nodes. The interconnection can have a tunable numeric weight that can be tuned (e.g., based on a training dataset), allowing the neural network architecture 500 to be adaptive to inputs and able to learn as more and more data is processed.

The neural network architecture 500 is pre-trained to process the features from the data in the input layer 520 using the different hidden layers 522a, 522b, through 522n in order to provide the output through the output layer 521.

In some cases, the neural network architecture 500 can adjust the weights of the nodes using a training process called backpropagation. A backpropagation process can include a forward pass, a loss function, a backward pass, and a weight update. The forward pass, loss function, backward pass, and parameter/weight update is performed for one training iteration. The process can be repeated for a certain number of iterations for each set of training data until the neural network architecture 500 is trained well enough so that the weights of the layers are accurately tuned.

To perform training, a loss function can be used to analyze an error in the output. Any suitable loss function definition can be used, such as a Cross-Entropy loss. Another example of a loss function includes the mean squared error (MSE), defined as E_total=σ(1/2 (target-output)\hat{ }2). The loss can be set to be equal to the value of E_total.

The loss (or error) will be high for the initial training data since the actual values will be much different than the predicted output. The goal of training is to minimize the amount of loss so that the predicted output is the same as the training output. The neural network architecture 500 can perform a backward pass by determining which inputs (weights) most contributed to the loss of the network and can adjust the weights so that the loss decreases and is eventually minimized.

The neural network architecture 500 can include any suitable deep network. One example includes a Convolutional Neural Network (CNN), which includes an input layer and an output layer, with multiple hidden layers between the input and out layers. The hidden layers of a CNN include a series of convolutional, nonlinear, pooling (for downsampling), and fully connected layers. The neural network architecture 500 can include any other deep network other than a CNN, such as an autoencoder, Deep Belief Nets (DBNs), Recurrent Neural Networks (RNNs), among others.

As understood by those of skill in the art, machine-learning based techniques can vary depending on the desired implementation. For example, machine-learning schemes can utilize one or more of the following, alone or in combination: hidden Markov models; RNNs; CNNs; deep learning; Bayesian symbolic methods; Generative Adversarial Networks (GANs); support vector machines; image registration methods; and applicable rule-based systems. Where regression algorithms are used, they may include but are not limited to: a Stochastic Gradient Descent Regressor, a Passive Aggressive Regressor, etc.

Machine learning classification models can also be based on clustering algorithms (e.g., a Mini-batch K-means clustering algorithm), a recommendation algorithm (e.g., a Minwise Hashing algorithm, or Euclidean Locality-Sensitive Hashing (LSH) algorithm), and/or an anomaly detection algorithm, such as a local outlier factor. Additionally, machine-learning models can employ a dimensionality reduction approach, such as, one or more of: a Mini-batch Dictionary Learning algorithm, an incremental Principal Component Analysis (PCA) algorithm, a Latent Dirichlet Allocation algorithm, and/or a Mini-batch K-means algorithm, etc.

### Example Computer System

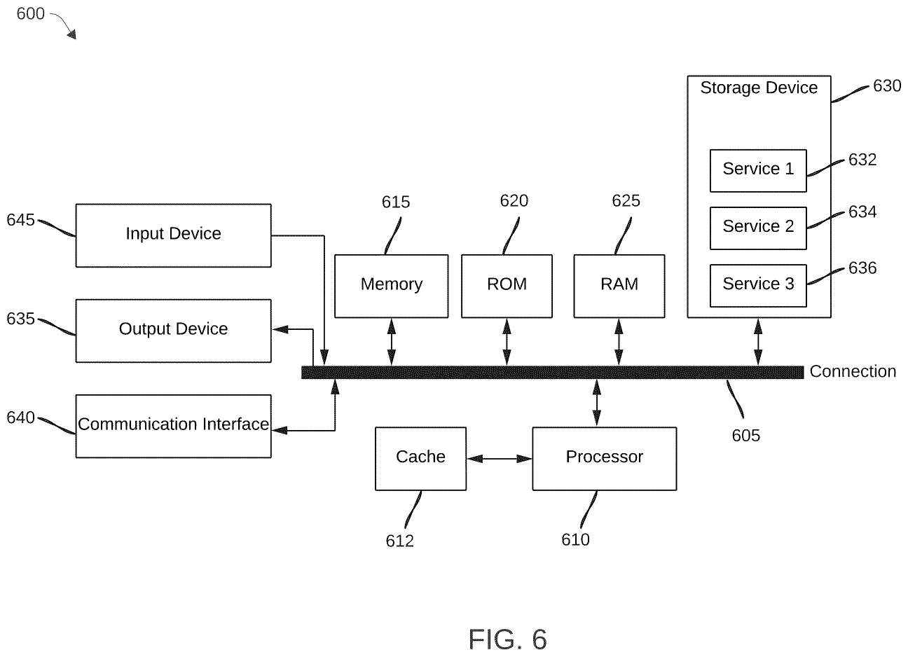

Various aspects and examples may be implemented, for example, using one or more well-known computer systems, such as computer system 600 shown in FIG. 6. For example, the smart scale 100 may be implemented using combinations or sub-combinations of computer system 600. Also, or alternatively, one or more computer systems 600 may be used, for example, to implement any of the aspects and examples discussed herein, as well as combinations and sub-combinations thereof.

Computer system 600 may include one or more processors (also called central processing units, or CPUs), such as a processor 604. Processor 604 may be connected to a communication infrastructure or bus 606.

Computer system 600 may also include user input/output device(s) 603, such as monitors, keyboards, pointing devices, etc., which may communicate with communication infrastructure 606 through user input/output interface(s) 602.

One or more of processors 604 may be a graphics processing unit (GPU). In some examples, a GPU may be a processor that is a specialized electronic circuit designed to process mathematically intensive applications. The GPU may have a parallel structure that is efficient for parallel processing of large blocks of data, such as mathematically intensive data common to computer graphics applications, images, videos, etc.

Computer system 600 may also include a main or primary memory 608, such as random-access memory (RAM). Main memory 608 may include one or more levels of cache. Main memory 608 may have stored therein control logic (e.g., computer software) and/or data.

Computer system 600 may also include one or more secondary storage devices or memory 610. Secondary memory 610 may include, for example, a hard disk drive 612 and/or a removable storage device or drive 614. Removable storage drive 614 may be a floppy disk drive, a magnetic tape drive, a compact disk drive, an optical storage device, tape backup device, and/or any other storage device/drive.

Removable storage drive 614 may interact with a removable storage unit 618. Removable storage unit 618 may include a computer usable or readable storage device having stored thereon computer software (control logic) and/or data. Removable storage unit 618 may be a floppy disk, magnetic tape, compact disk, DVD, optical storage disk, and/any other computer data storage device. Removable storage drive 614 may read from and/or write to removable storage unit 618.

Secondary memory 610 may include other means, devices, components, instrumentalities or other approaches for allowing computer programs and/or other instructions and/or data to be accessed by computer system 600. Such means, devices, components, instrumentalities or other approaches may include, for example, a removable storage unit 622 and an interface 620. Examples of the removable storage unit 622 and the interface 620 may include a program cartridge and cartridge interface (such as that found in video game devices), a removable memory chip (such as an EPROM or PROM) and associated socket, a memory stick and USB or other port, a memory card and associated memory card slot, and/or any other removable storage unit and associated interface.

Computer system 600 may include a communication or network interface 624. Communication interface 624 may enable computer system 600 to communicate and interact with any combination of external devices, external networks, external entities, etc. (individually and collectively referenced by reference number 628). For example, communication interface 624 may allow computer system xx00 to communicate with external or remote devices 628 over communications path 626, which may be wired and/or wireless (or a combination thereof), and which may include any combination of LANs, WANs, the Internet, etc. Control logic and/or data may be transmitted to and from computer system 600 via communication path 626.

Computer system 600 may also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 600 may be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 600 may be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas may be used, either exclusively or in combination with known or open standards.

In some examples, a tangible, non-transitory apparatus or article of manufacture comprising a tangible, non-transitory computer useable or readable medium having control logic (software) stored thereon may also be referred to herein as a computer program product or program storage device. This includes, but is not limited to, computer system 600, main memory 608, secondary memory 610, and removable storage units 618 and 622, as well as tangible articles of manufacture embodying any combination of the foregoing. Such control logic, when executed by one or more data processing devices (such as computer system 600 or processor(s) 604), may cause such data processing devices to operate as described herein.

Based on the teachings contained in this disclosure, it will be apparent to persons skilled in the relevant art(s) how to make and use embodiments of this disclosure using data processing devices, computer systems and/or computer architectures other than that shown in FIG. 6. In particular, embodiments can operate with software, hardware, and/or operating system implementations other than those described herein.

### Conclusion

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

While this disclosure describes exemplary embodiments for exemplary fields and applications, it should be understood that the disclosure is not limited thereto. Other embodiments and modifications thereto are possible, and are within the scope and spirit of this disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments can perform functional blocks, steps, operations, methods, etc. using orderings different than those described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but yet still co-operate or interact with each other.

The breadth and scope of this disclosure should not be limited by any of the above-described exemplary embodiments, but should be defined only in accordance with the following claims and their equivalents.

Claim language or other language in the disclosure reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

Illustrative examples of the disclosure include:
Aspect 1. A computer-implemented method comprising: obtaining sensor data from one or more sensors of a smart scale; determining at least a first health metric based on the sensor data; determining a color associated with the first health metric based on color map data associated with the first health metric; and emitting a light, via a display system, in accordance with the color associated with the first health metric, the light being emitted towards one or more portions of a platform of the smart scale and causing the platform to emit light in the color associated with the first health metric.
Aspect 2. The computer-implemented method of Aspect 1, wherein determining the color associated with the first health metric includes: determining a color map associated with the first health metric based on a type of health metric of the first health metric and the color map data, each color identified in the color map is associated with a range of health metric values; and determining a value of the first health metric is within a range of health metric values associated with the color.
Aspect 3. The computer-implemented method of any of Aspects 1 to 2, further comprising: obtaining an electrical signal from one or more electrodes of a smart scale; wherein the first health metric is further based on the electrical signal.
Aspect 4. The computer-implemented method of Aspect 3, further comprising: determining a second health metric based on the sensor data and the electrical signal; determining a color associated with the second health metric based on color map data associated with the second health metric; and emitting, by a first portion of the display system, light in accordance with the color associated with the first health metric, and, by a second portion of the display system, light in accordance with the color associated with the second health metric.
Aspect 5. The computer-implemented method of Aspect 4, wherein, the light emitted by the first portion of the display system is emitted to a first portion of the platform and the light emitted by the second portion of the display system is emitted to a second portion of the platform, the first portion of the platform emits the light in the color associated with the first health metric and the second portion of the platform emits the light in the color associated with the second health metric.
Aspect 6. The computer-implemented method of any of Aspects 1 to 5, further comprising: obtaining historical health information associated with the first health metric; wherein determining the color associated with the first health metric is based on the historical health information and the color map data associated with the first health metric, the color representing the first health metric over a period of time.
Aspect 7. The computer-implemented method of any of Aspects 1 to 6, wherein the display system includes a display device, and wherein the computer-implemented method further comprises: causing the display device to output a numerical value associated with the first health metric.
Aspect 8. A smart scale comprising: a platform; a display system; a memory storing instructions; and at least one processor coupled to the memory and configured to execute the instructions to: obtain sensor data from one or more sensors of a smart scale; determine at least a first health metric based on the sensor data; determine a color associated with the first health metric based on color map data associated with the first health metric; and emit a light, via the display system, in accordance with the color associated with the first health metric, the light being emitted towards one or more portions of the platform of the smart scale and causing the platform to emit light in the color associated with the first health metric.
Aspect 9. The smart scale of Aspect 8, wherein to determine the color associated with the first health metric, the at least one processor is configured to execute the instructions to: determine a color map associated with the first health metric based on a type of health metric of the first health metric and the color map data, each color identified in the color map is associated with a range of health metric values; and determine a value of the first health metric is within a range of health metric values associated with the color.
Aspect 10. The smart scale of any of Aspects 8 to 9, wherein the at least one processor is configured to execute the instructions further to: obtain an electrical signal from one or more electrodes of a smart scale; wherein the first health metric is further based on the electrical signal.
Aspect 11. The smart scale of Aspect 10, wherein the at least one processor is configured to execute the instructions further to: determine a second health metric based on the sensor data and the electrical signal; determine a color associated with the second health metric based on color map data associated with the second health metric; and emit, by a first portion of the display system, light in accordance with the color associated with the first health metric, and, by a second portion of the display system, light in accordance with the color associated with the second health metric.
Aspect 12. The smart scale of Aspect 11, wherein, the light emitted by the first portion of the display system is emitted to a first portion of the platform and the light emitted by the second portion of the display system is emitted to a second portion of the platform, the first portion of the platform emits the light in the color associated with the first health metric and the second portion of the platform emits the light in the color associated with the second health metric.
Aspect 13. The smart scale of any of Aspects 8 to 12, wherein the at least one processor is configured to execute the instructions further to: obtain historical health information associated with the first health metric; wherein determining the color associated with the first health metric is based on the historical health information and the color map data associated with the first health metric, the color representing the first health metric over a period of time.
Aspect 14. The smart scale of any of Aspects 8 to 13, wherein the display system includes a display device, and wherein the at least one processor is configured to execute the instructions further to: cause the display device to output a numerical value associated with the first health metric.
Aspect 15. A non-transitory computer-readable medium having instructions stored thereon that, when executed by at least one computing device, cause the at least one computing device to perform operations comprising: obtaining sensor data from one or more sensors of a smart scale; determining at least a first health metric based on the sensor data; determining a color associated with the first health metric based on color map data associated with the first health metric; and emitting a light, via a display system, in accordance with the color associated with the first health metric, the light being emitted towards one or more portions of a platform of the smart scale and causing the platform to emit light in the color associated with the first health metric.
Aspect 16. The non-transitory computer-readable medium of Aspect 15, wherein determining the color associated with the first health metric includes: determining a color map associated with the first health metric based on a type of health metric of the first health metric and the color map data, each color identified in the color map is associated with a range of health metric values; and determining a value of the first health metric is within a range of health metric values associated with the color.
Aspect 17. The non-transitory computer-readable medium of any of Aspects 15 to 16, wherein the at least one computing device further performs operations comprising: obtaining an electrical signal from one or more electrodes of a smart scale; wherein the first health metric is further based on the electrical signal.
Aspect 18. The non-transitory computer-readable medium of Aspect 17, wherein the at least one computing device further performs operations comprising: determining a second health metric based on the sensor data and the electrical signal; determining a color associated with the second health metric based on color map data associated with the second health metric; and emitting, by a first portion of the display system, light in accordance with the color associated with the first health metric, and, by a second portion of the display system, light in accordance with the color associated with the second health metric.
Aspect 19. The non-transitory computer-readable medium of Aspect 18, wherein, the light emitted by the first portion of the display system is emitted to a first portion of the platform and the light emitted by the second portion of the display system is emitted to a second portion of the platform, the first portion of the platform emits the light in the color associated with the first health metric and the second portion of the platform emits the light in the color associated with the second health metric.
Aspect 20. The non-transitory computer-readable medium of any of Aspects 15 to 19, wherein the display system includes a display device, and wherein the at least one computing device further performs operations comprising: causing the display device to output a numerical value associated with the first health metric.

What is claimed is:

1. A computer-implemented method comprising:
obtaining sensor data from one or more sensors of a smart scale;
determining at least a first health metric based on the sensor data;
determining a color associated with the first health metric based on color map data associated with the first health metric; and
emitting a light, via a display system, in accordance with the color associated with the first health metric, the light being emitted towards one or more portions of a platform of the smart scale and causing the platform to emit light in the color associated with the first health metric.

2. The computer-implemented method of claim 1, wherein determining the color associated with the first health metric includes:
determining a color map associated with the first health metric based on a type of health metric of the first health metric and the color map data, each color identified in the color map is associated with a range of health metric values; and
determining a value of the first health metric is within a range of health metric values associated with the color.

3. The computer-implemented method of claim 1, further comprising:
obtaining an electrical signal from one or more electrodes of a smart scale;
wherein the first health metric is further based on the electrical signal.

4. The computer-implemented method of claim 3, further comprising:
determining a second health metric based on the sensor data and the electrical signal;
determining a color associated with the second health metric based on color map data associated with the second health metric; and
emitting, by a first portion of the display system, light in accordance with the color associated with the first health metric, and, by a second portion of the display system, light in accordance with the color associated with the second health metric.

5. The computer-implemented method of claim 4, wherein, the light emitted by the first portion of the display system is emitted to a first portion of the platform and the light emitted by the second portion of the display system is emitted to a second portion of the platform, the first portion of the platform emits the light in the color associated with the first health metric and the second portion of the platform emits the light in the color associated with the second health metric.

6. The computer-implemented method of claim 1, further comprising:
obtaining historical health information associated with the first health metric;
wherein determining the color associated with the first health metric is based on the historical health information and the color map data associated with the first health metric, the color representing the first health metric over a period of time.

7. The computer-implemented method of claim 1, wherein the display system includes a display device, and wherein the computer-implemented method further comprises:
causing the display device to output a numerical value associated with the first health metric.

8. A smart scale comprising:
a platform;
a display system;
a memory storing instructions; and
at least one processor coupled to the memory and configured to execute the instructions to:
obtain sensor data from one or more sensors of a smart scale;
determine at least a first health metric based on the sensor data;
determine a color associated with the first health metric based on color map data associated with the first health metric; and
emit a light, via the display system, in accordance with the color associated with the first health metric, the light being emitted towards one or more portions of the platform of the smart scale and causing the platform to emit light in the color associated with the first health metric.

9. The smart scale of claim 8, wherein to determine the color associated with the first health metric, the at least one processor is configured to execute the instructions to:
determine a color map associated with the first health metric based on a type of health metric of the first health metric and the color map data, each color identified in the color map is associated with a range of health metric values; and
determine a value of the first health metric is within a range of health metric values associated with the color.

10. The smart scale of claim 8, wherein the at least one processor is configured to execute the instructions further to:
obtain an electrical signal from one or more electrodes of a smart scale;
wherein the first health metric is further based on the electrical signal.

11. The smart scale of claim 10, wherein the at least one processor is configured to execute the instructions further to:
determine a second health metric based on the sensor data and the electrical signal;
determine a color associated with the second health metric based on color map data associated with the second health metric; and
emit, by a first portion of the display system, light in accordance with the color associated with the first health metric, and, by a second portion of the display system, light in accordance with the color associated with the second health metric.

12. The smart scale of claim 11, wherein, the light emitted by the first portion of the display system is emitted to a first portion of the platform and the light emitted by the second portion of the display system is emitted to a second portion of the platform, the first portion of the platform emits the light in the color associated with the first health metric and the second portion of the platform emits the light in the color associated with the second health metric.

13. The smart scale of claim 8, wherein the at least one processor is configured to execute the instructions further to:
obtain historical health information associated with the first health metric;
wherein determining the color associated with the first health metric is based on the historical health information and the color map data associated with the first health metric, the color representing the first health metric over a period of time.

14. The smart scale of claim 8, wherein the display system includes a display device, and wherein the at least one processor is configured to execute the instructions further to:
cause the display device to output a numerical value associated with the first health metric.

15. A non-transitory computer-readable medium having instructions stored thereon that, when executed by at least one computing device, cause the at least one computing device to perform operations comprising:
obtaining sensor data from one or more sensors of a smart scale;
determining at least a first health metric based on the sensor data;
determining a color associated with the first health metric based on color map data associated with the first health metric; and
emitting a light, via a display system, in accordance with the color associated with the first health metric, the light being emitted towards one or more portions of a platform of the smart scale and causing the platform to emit light in the color associated with the first health metric.

16. The non-transitory computer-readable medium of claim 15, wherein determining the color associated with the first health metric includes:
determining a color map associated with the first health metric based on a type of health metric of the first health metric and the color map data, each color identified in the color map is associated with a range of health metric values; and
determining a value of the first health metric is within a range of health metric values associated with the color.

17. The non-transitory computer-readable medium of claim 15, wherein the at least one computing device further performs operations comprising:
obtaining an electrical signal from one or more electrodes of a smart scale;
wherein the first health metric is further based on the electrical signal.

18. The non-transitory computer-readable medium of claim 17, wherein the at least one computing device further performs operations comprising:
determining a second health metric based on the sensor data and the electrical signal;
determining a color associated with the second health metric based on color map data associated with the second health metric; and
emitting, by a first portion of the display system, light in accordance with the color associated with the first health metric, and, by a second portion of the display system, light in accordance with the color associated with the second health metric.

19. The non-transitory computer-readable medium of claim 18, wherein, the light emitted by the first portion of the display system is emitted to a first portion of the platform and the light emitted by the second portion of the display system is emitted to a second portion of the platform, the first portion of the platform emits the light in the color associated with the first health metric and the second portion of the platform emits the light in the color associated with the second health metric.

20. The non-transitory computer-readable medium of claim 15, wherein the display system includes a display device, and wherein the at least one computing device further performs operations comprising:
causing the display device to output a numerical value associated with the first health metric.
